(19) United States
(12) Patent Application Publication
(10) Pub. No.: US 2025/0334880 A1
(43) Pub. Date: Oct. 30, 2025

(54) RADIATION-SENSITIVE COMPOSITION, METHOD FOR FORMING RESIST PATTERN, AND COMPOUND

(71) Applicant: JSR CORPORATION, Tokyo (JP)

(72) Inventors: Katsuaki NISHIKORI, Tokyo (JP); Yasuhiro OHTAGAKI, Tokyo (JP)

(73) Assignee: JSR CORPORATION, Tokyo (JP)

(21) Appl. No.: 19/264,990

(22) Filed: Jul. 10, 2025

Related U.S. Application Data
(63) Continuation of application No. PCT/JP2023/042975, filed on Nov. 30, 2023.

(30) Foreign Application Priority Data
Jan. 13, 2023 (JP) 2023-004072

(51) Int. Cl. G03F 7/039

(52) U.S. Cl. CPC G03F 7/039

(57) ABSTRACT

A radiation-sensitive composition contains: a polymer, solubility of which in a developer solution is capable of being altered by an action of an acid; an anion represented by formula (1); and a radiation-sensitive onium cation containing an aromatic ring and at least one fluorine atom or fluorine atom-containing group bonded to the aromatic ring. Ar¹ represents a group obtained by removing one hydrogen atom from a substituted or unsubstituted aromatic ring; n is an integer of 1 to 3; R¹ represents a single bond or a substituted or unsubstituted divalent hydrocarbon group; L¹ represents —O—, (*)ₙ—R²—O—, or —NR³—, wherein in a case in which n is no less than 2, L¹ represents (*)ₙ—R²—O—; * denotes a site bonding to Ar¹; R² represents a substituted or unsubstituted hydrocarbon group having a valency of (n+1); and R³ represents a hydrogen atom or a monovalent hydrocarbon group.

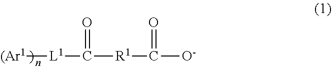

### CROSS-REFERENCE TO RELATED APPLICATIONS

The present application is a continuation application of International Patent Application No. PCT/JP2023/042975 filed Nov. 30, 2023, which claims priority to Japanese Patent Application No. 2023-004072 filed Jan. 13, 2023. The contents of these applications are incorporated herein by reference in their entirety.

### BACKGROUND OF THE DISCLOSURE

### Technical Field

The present disclosure relates to a radiation-sensitive composition, a method of forming a resist pattern, and a compound.

### Discussion of the Background

A radiation-sensitive composition for use in microfabrication by lithography generates an acid at light-exposed regions upon an irradiation with a radioactive ray, e.g., an electromagnetic wave such as a far ultraviolet ray such as an ArF excimer laser beam (wavelength of 193 nm), a KrF excimer laser beam (wavelength of 248 nm), etc. or an extreme ultraviolet ray (EUV) (wavelength of 13.5 nm), or a charged particle ray such as an electron beam. A chemical reaction that originates from the acid causes a difference between the light-exposed regions and light-unexposed regions in rates of dissolution in a developer solution, whereby a resist pattern is formed on a substrate.

Such a radiation-sensitive composition is required not only to have favorable sensitivity to a radioactive ray such as an extreme ultraviolet ray and an electron beam, but also to have superiority in terms of CDU (critical dimension uniformity) and the like.

Types, molecular structures, and the like of polymers, acid generating agents, and other components which may be used in radiation-sensitive compositions have been investigated to meet these requirements, and combinations thereof have been further investigated in detail (see Japanese Unexamined Patent Applications, Publication Nos. 2010-134279, 2014-224984, 2016-047815, and 2021-009357).

### SUMMARY

According to an aspect of the present disclosure, a radiation-sensitive composition includes: a polymer, solubility of which in a developer solution is capable of being altered by an action of an acid; an anion represented by formula (1); and a radiation-sensitive onium cation including an aromatic ring and at least one fluorine atom or fluorine atom-containing group bonded to the aromatic ring.

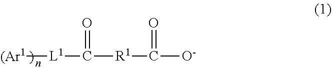

In the formula (1), Ar¹ represents a group obtained by removing one hydrogen atom from a substituted or unsubstituted aromatic ring; n is an integer of 1 to 3; in a case in which n is no less than 2, a plurality of Aris are identical or different; R¹ represents a single bond or a substituted or unsubstituted divalent hydrocarbon group; L¹ represents —O—, (*)ₙ—R²—O—, or —NR³—, wherein in a case in which n is no less than 2, L¹ represents (*)ₙ—R²—O—; * denotes a site bonding to Ar¹; R² represents a substituted or unsubstituted hydrocarbon group having a valency of (n+1); and R³ represents a hydrogen atom or a monovalent hydrocarbon group.

According to another aspect of the present disclosure, a method of forming a resist pattern, includes: applying the above-described radiation-sensitive composition directly or indirectly on a substrate to form a resist film to form a resist film; exposing the resist film; and developing the resist film exposed.

According to a further aspect of the present disclosure, a compound is represented by formula (2).

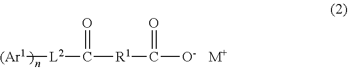

In the formula (2), Ar¹ represents a group obtained by removing one hydrogen atom from a substituted or unsubstituted aromatic ring; n is an integer of 1 to 3; in a case in which n is no less than 2, a plurality of Aris are identical or different; R¹ represents a single bond or a substituted or unsubstituted divalent hydrocarbon group; L² represents (*)ₙ—R²—O—; R² represents a substituted or unsubstituted hydrocarbon group having a valency of (n+1); * denotes a site bonding to Ar¹; and M⁺ represents a monovalent cation.

### DESCRIPTION OF THE EMBODIMENTS

The present inventors have newly found that when a radiation-sensitive composition containing: a radiation-sensitive onium cation containing an aromatic ring having at least one fluorine atom or fluorine atom-containing group bonded to the aromatic ring; and an aromatic carboxylic acid anion such as a benzoic acid anion or a salicylic acid anion is used in an attempt to improve the sensitivity and the CDU of the radiation-sensitive composition, lithography performance such as the sensitivity and the CDU is improved, but the sensitivity changes over time (see Comparative Examples 1 to 2 described later). Hereinafter, the degree of the change in the sensitivity over time is referred to as “storage stability”. The smaller the change in the sensitivity over time, the more superior the storage stability.

As described above, it has been required to balance the lithography performance such as the sensitivity and the CDU, and the storage stability.

Although restrictive interpretation is not intended, the present inventors have believed that the storage stability deteriorates due to high basicity of the aromatic carboxylic acid anion in the radiation-sensitive composition, and have found that the lithography performance such as the sensitivity and the CDU, and the storage stability can be balanced when an anion having lower basicity than the aromatic carboxylic acid anion is adopted. Furthermore, the present inventors have found that the effect of the aromatic carboxylic acid anion on the storage stability is a characteristic problem that arises in the case in which the radiation-sensitive onium cation containing the aromatic ring having at least one fluorine atom or fluorine atom-containing group bonded to the aromatic ring is contained.

An embodiment of the present disclosure is a radiation-sensitive composition containing: a polymer (hereinafter, may be also referred to as “(A) polymer” or “polymer (A)”), solubility of which in a developer solution is capable of being altered by an action of an acid; an anion (hereinafter, may be also referred to as “(X) anion” or “anion (X)”) represented by the following formula (1); and a radiation-sensitive onium cation (hereinafter, may be also referred to as “(Y) cation” or “cation (Y)”) containing an aromatic ring having at least one fluorine atom or fluorine atom-containing group bonded to the aromatic ring.

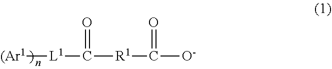

In the formula (1), Ar¹ represents a group obtained by removing one hydrogen atom from a substituted or unsubstituted aromatic ring; n is an integer of 1 to 3; in a case in which n is no less than 2, a plurality of Aris are identical or different; R¹ represents a single bond or a substituted or unsubstituted divalent hydrocarbon group; L¹ represents —O—, (*)ₙ—R²—O—, or —NR³—, wherein in a case in which n is no less than 2, L¹ represents (*)ₙ—R²—O—; * denotes a site bonding to Ar¹; R² represents a substituted or unsubstituted hydrocarbon group having a valency of (n+1); and R³ represents a hydrogen atom or a monovalent hydrocarbon group.

Another embodiment of the present disclosure is a method of forming a resist pattern. The method includes: applying the above-described radiation-sensitive composition directly or indirectly on a substrate to form a resist film; exposing the resist film; and developing the resist film exposed.

Yet another embodiment of the present disclosure is a compound represented by the following formula (2).

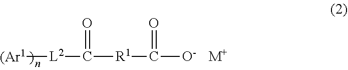

In the formula (2), Ar¹ represents a group obtained by removing one hydrogen atom from a substituted or unsubstituted aromatic ring; n is an integer of 1 to 3, wherein in a case in which n is no less than 2, a plurality of Aris are identical or different; R¹ represents a single bond or a substituted or unsubstituted divalent hydrocarbon group; L² represents (*)ₙ—R²—O—; R² represents a substituted or unsubstituted hydrocarbon group having a valency of (n+1); * denotes a site bonding to Ar¹; and M⁺ represents a monovalent cation.

The radiation-sensitive composition of the present disclosure is superior in sensitivity, CDU, and storage stability. The method of forming a resist pattern of the present disclosure enables favorably forming a resist pattern because the sensitivity is favorable, the CDU is superior, and a change in the sensitivity over time is inhibited. The compound of the present disclosure can be suitably used as a component of a radiation-sensitive composition being superior in sensitivity, CDU, and storage stability. Hereinafter, the radiation-sensitive composition, the method of forming a resist pattern, and the compound of the present disclosure will be described in detail.

With respect to descriptions of the upper limit and the lower limit of numerical ranges as referred to herein, unless otherwise specified particularly, the upper limit may have the meaning of either “no greater than” or “less than”, and the lower limit may have the meaning of either “no less than” or “greater than”. Further, as the upper limit value and the lower limit value, disclosed numerical values may be combined ad libitum. Furthermore, in a case in which a numerical range is shown using the word “to”, the numerical range is intended to include the upper limit numerical value and the lower limit numerical value. For example, the phrase “1 to 20 carbon atoms” as referred to herein means “1 or more and 20 or less carbon atoms”.

Further, as used herein, the words “a” and “an” and the like carry the meaning of “one or more.” When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed. Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

### Radiation-Sensitive Composition

The radiation-sensitive composition contains the polymer (A), the anion (X), and the cation (Y). The anion (X) is derived from a compound containing the anion (X), and the cation (Y) is derived from a compound containing the cation (Y). The anion (X) and the cation (Y) may be derived from different compounds, or may be derived from an identical compound, i.e., may be derived from a compound containing the anion (X) and the cation (Y).

The radiation-sensitive composition typically contains an organic solvent (hereinafter, may be also referred to as “(D) organic solvent” or “organic solvent (D)”).

The radiation-sensitive composition may contain a radiation-sensitive acid generating agent (hereinafter, may be also referred to as “(B) acid generating agent” or “acid generating agent (B)”) as a suitable component. The acid generating agent (B) is not particularly limited as long as it is a compound that can serve as a radiation-sensitive acid generating agent. For example, the acid generating agent (B) may be the compound containing the cation (Y), or may be the compound not containing the cation (Y).

The radiation-sensitive composition may contain an acid diffusion control agent (hereinafter, may be also referred to as “(C) acid diffusion control agent” or “acid diffusion control agent (C)”). The acid diffusion control agent (C) is not particularly limited as long as it is a compound that can serve as an acid diffusion control agent. The acid diffusion control agent (C) may be the compound containing the anion (X), may be the compound containing the anion (X) and the cation (Y), or may be the compound not containing the anion (X).

The radiation-sensitive composition may contain a polymer (hereinafter, may be also referred to as “(F) polymer” or “polymer (F)”) having a percentage content of fluorine atoms higher than that of the polymer (A). The radiation-sensitive composition can contain, within a range not leading to impairment of the effects of the present invention, other optional component(s).

Owing to containing the polymer (A), the anion (X), and the cation (Y), the radiation-sensitive composition is superior in sensitivity, CDU, and storage stability. Although not necessarily clarified and without wishing to be bound by any theory, the reason for achieving the above-described effects by the radiation-sensitive composition due to involving such a constitution may be presumed, for example, as in the following. It is believed that: containing the cation (Y) improves absorption efficiency of a radioactive ray, whereby the effective amount of the generated acid increases, and thus the sensitivity and the CDU improve; and containing the anion (X) decreases the basicity of the anion, and thus the storage stability improves.

The radiation-sensitive composition can be prepared, for example, by: mixing, in a certain ratio, the polymer (A), the compound containing the anion (X), and the compound containing the cation (Y), as well as the acid generating agent (B), the acid diffusion control agent (C), the organic solvent (D), the polymer (F), the other optional component(s), and the like, which are added as needed; and filtering a thus resulting mixture through a membrane filter having a pore size of no greater than 0.2 μm.

Each component contained in the radiation-sensitive composition is described below.

### (A) Polymer

The polymer (A) is a polymer, solubility of which in a developer solution is capable of being altered by an action of an acid. In general, when the polymer (A) contains an acid-labile group, the polymer (A) exhibits the property of enabling the solubility in a developer solution to be altered by an action of an acid. Therefore, the polymer (A) preferably has a structural unit (hereinafter, may be also referred to as “structural unit (I)”) containing an acid-labile group. The radiation-sensitive composition can contain one, or two or more types of the polymer (A).

The polymer (A) preferably further has a structural unit (hereinafter, may be also referred to as “structural unit (II)”) containing a phenolic hydroxyl group. The polymer (A) may further have a structural unit (hereinafter, may be also simply referred to as “other structural unit”) other than the structural unit (I) and the structural unit (II). The polymer (A) can have one, or two or more types of each structural unit.

The lower limit of a proportion of the polymer (A) in the radiation-sensitive composition with respect to total components, other than the organic solvent (D), contained in the radiation-sensitive composition is preferably 50% by mass, more preferably 70% by mass, and still more preferably 80% by mass. The upper limit of the proportion is preferably 99% by mass, and more preferably 95% by mass.

The lower limit of a polystyrene-equivalent weight average molecular weight (Mw) of the polymer (A) as determined by gel permeation chromatography (GPC) is preferably 1,000, more preferably 2,000, still more preferably 3,000, and even further preferably 5,000. The upper limit of the Mw is preferably 30,000, more preferably 20,000, still more preferably 10,000, and even further preferably 7,000. When the Mw of the polymer (A) falls within the above range, coating characteristics of the radiation-sensitive composition may be improved. The Mw of the polymer (A) can be adjusted by, for example, regulating the type, the using amount, and the like of a polymerization initiator used in synthesis of the polymer (A).

The lower limit of a ratio (hereinafter, may be also referred to as “Mw/Mn” or “polydispersity index”) of the Mw to a polystyrene-equivalent number average molecular weight (Mn) of the polymer (A) as determined by GPC is typically 1.0, preferably 1.1, more preferably 1.2, still more preferably 1.3, and even further preferably 1.4. The upper limit of the ratio is preferably 2.5, more preferably 2.0, still more preferably 1.8, and even further preferably 1.7.

### Method for Measuring Mw and Mn

As referred to herein, the Mw and Mn of the polymers are values measured by using gel permeation chromatography (GPC) under the following conditions.
GPC columns: “G2000 HXL”×2, “G3000 HXL”×1, and “G4000 HXL”×1, each available from Tosoh Corporation
column temperature: 40° C.
elution solvent: tetrahydrofuran
flow rate: 1.0 mL/min
sample concentration: 1.0% by mass
amount of injected sample: 100 uL
detector: differential refractometer
standard substance: mono-dispersed polystyrene

The polymer (A) can be synthesized by, for example, polymerizing a monomer that gives each structural unit in accordance with a well-known procedure.

Each structural unit included in the polymer (A) is described below.

### Structural Unit (I)

The structural unit (I) is a structural unit containing an acid-labile group. The term “acid-labile group” as referred to herein means a group that substitutes for a hydrogen atom in a carboxy group or a hydrogen atom in a hydroxy group, and is capable of being dissociated by an action of an acid to give a carboxy group or a hydroxy group. More specifically, the structural unit (I) is a structural unit including a partial structure obtained by substituting a hydrogen atom of a carboxy group or a hydrogen atom of a phenolic hydroxyl group, with an acid-labile group. It is to be noted that herein, a structural unit containing both an acid-labile group and a phenolic hydroxyl group is encompassed by the structural unit (II) described later.

Owing to containing an acid-labile group, the polymer (A) exhibits the property of enabling the solubility in a developer solution to be altered by an action of an acid. The acid-labile group is dissociated by an action of the acid generated from the acid generating agent (B), etc. upon exposure, whereby a difference is generated in the solubility of the polymer (A) in the developer solution, between light-exposed regions and light-unexposed regions, and thus forming a resist pattern is enabled.

The acid-labile group is a group that substitutes for a hydrogen atom included in a carboxy group or a hydrogen atom included in a phenolic hydroxyl group in the structural unit (I). In other words, in the structural unit (I), the acid-labile group bonds to an ethereal oxygen atom of a carbonyloxy group or to an oxygen atom of a phenolic hydroxyl group.

The acid-labile group is exemplified by groups (hereinafter, may be also referred to as “acid-labile groups (a-1) to (a-3)”) represented by the following formulae (a-1) to (a-3).

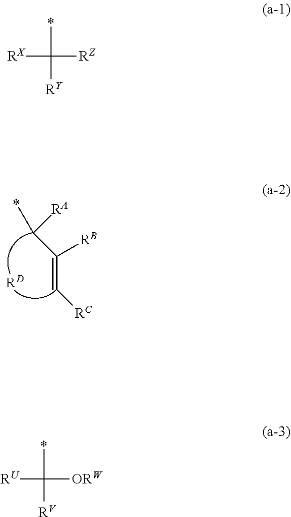

In the above formulae (a-1) to (a-3), * denotes a site bonding to the ethereal oxygen atom of the carboxy group or to the oxygen atom of the phenolic hydroxyl group.

In the above formula (a-1), RX represents a substituted or unsubstituted monovalent hydrocarbon group having 1 to 20 carbon atoms; and R^{Y} and R^{Z} each independently represent a monovalent hydrocarbon group having 1 to 20 carbon atoms, or R^{Y} and R^{Z} taken together represent a saturated alicyclic ring having 3 to 20 ring atoms, together with the carbon atom to which R^{Y} and R^{Z} bond.

In the above formula (a-2), R^{A} represents a hydrogen atom; R^{B} and R^{C} each independently represent a hydrogen atom or a monovalent hydrocarbon group having 1 to 20 carbon atoms; and R^{D} represents a divalent hydrocarbon group having 1 to 20 carbon atoms, and constituting an unsaturated alicyclic ring having 4 to 20 ring atoms, together with the three carbon atoms to which R^{A}, R^{B}, and R¹ bond, respectively.

In the above formula (a-3), R^{U} and R^{V} each independently represent a hydrogen atom or a monovalent hydrocarbon group having 1 to 20 carbon atoms, and R^{W} represents a monovalent hydrocarbon group having 1 to 20 carbon atoms; R^{U} and R^{V} taken together represent a saturated alicyclic ring having 3 to 20 ring atoms, together with the carbon atom to which R^{U} and R^{V} bond; or R^{V} and R^{W} taken together represent an oxygen atom-containing aliphatic heterocyclic ring having 4 to 20 ring atoms, together with the carbon atom to which RV bonds, and the oxygen atom to which R^{W} bonds.

The number of “carbon atoms” as referred to herein means the number of carbon atoms constituting a group. The “hydrocarbon group” encompasses an “aliphatic hydrocarbon group” and an “aromatic hydrocarbon group”. The “aliphatic hydrocarbon group” encompasses a “chain hydrocarbon group” and an “alicyclic hydrocarbon group”. In another light, the “aliphatic hydrocarbon group” encompasses a “saturated hydrocarbon group” and an “unsaturated hydrocarbon group”. The “chain hydrocarbon group” as referred to herein means a hydrocarbon group not having a ring structure and being constituted by only a chain structure, and may be exemplified by both a linear hydrocarbon group and a branched hydrocarbon group. The “alicyclic hydrocarbon group” as referred to herein means a hydrocarbon group having, as a ring structure, not an aromatic ring but an alicyclic ring alone, and may be exemplified by both a monocyclic alicyclic hydrocarbon group and a polycyclic alicyclic hydrocarbon group. With regard to this, it is not necessary for the alicyclic hydrocarbon group to be constituted of only an alicyclic structure; it may have a chain structure in a part thereof. The “aromatic hydrocarbon group” as referred to herein means a hydrocarbon group that contains an aromatic ring as a ring structure. With regard to this, it is not necessary for the aromatic hydrocarbon group to be constituted of only an aromatic ring, and it may have a chain structure or an alicyclic ring in a part thereof.

The number of “ring atoms” as referred to herein means the number of atoms constituting a ring structure, and in the case of a polycyclic ring, the number of “ring atoms” means the number of atoms constituting the polycyclic ring. The “polycyclic ring” encompasses not only a spiro-type polycyclic ring in which two rings have one shared atom and a condensed polycyclic ring in which two rings have two shared atoms, but also a ring-assembled polycyclic ring in which two rings are connected by a single bond without having any shared atom.

The “ring structure” encompasses both an “alicyclic ring” and an “aromatic ring”. The “alicyclic ring” encompasses both an “aliphatic hydrocarbon ring” and an “aliphatic heterocyclic ring”. Of the alicyclic rings, a polycyclic one containing both the aliphatic hydrocarbon ring and the aliphatic heterocyclic ring falls under the “aliphatic heterocyclic ring”. The “aromatic ring” encompasses both an “aromatic hydrocarbon ring” and an “aromatic heterocyclic ring”. Of the aromatic rings, a polycyclic one containing both the aromatic hydrocarbon ring and the aromatic heterocyclic ring falls under the “aromatic heterocyclic ring”.

The monovalent hydrocarbon group that has 1 to 20 carbon atoms and may be represented by R^{X}, R^{Y}, R^{Z}, R^{B}, R^{C}, R^{U}, R^{V}, or R^{W} is exemplified by a monovalent chain hydrocarbon group having 1 to 20 carbon atoms, a monovalent alicyclic hydrocarbon group having 3 to 20 carbon atoms, and a monovalent aromatic hydrocarbon group having 6 to 20 carbon atoms.

The substituent that may be incorporated in the hydrocarbon group represented by R^{X} is exemplified by: halogen atoms such as a fluorine atom and an iodine atom; a hydroxy group; a carboxy group; a cyano group; a nitro group; an alkoxy group; an alkoxycarbonyl group; an alkoxycarbonyloxy group; an acyl group; and an acyloxy group. It is preferred that R^{X} contains an iodine atom as the substituent because more improving the CDU of the radiation-sensitive composition tends to be enabled.

Examples of the monovalent chain hydrocarbon group having 1 to 20 carbon atoms include: alkyl groups such as a methyl group, an ethyl group, an n-propyl group, an i-propyl group, an n-butyl group, a sec-butyl group, an isobutyl group, and a tert-butyl group; alkenyl groups such as an ethenyl group, a propenyl group, a butenyl group, and a 2-methylprop-1-en-1-yl group; and alkynyl groups such as an ethynyl group, a propynyl group, and a butynyl group.

Examples of the monovalent alicyclic hydrocarbon group having 3 to 20 carbon atoms include: monocyclic alicyclic saturated hydrocarbon groups such as a cyclopentyl group and a cyclohexyl group; polycyclic alicyclic saturated hydrocarbon groups such as a norbornyl group, an adamantyl group, a tricyclodecyl group, and a tetracyclododecyl group; monocyclic alicyclic unsaturated hydrocarbon groups such as a cyclopentenyl group and a cyclohexenyl group; and polycyclic alicyclic unsaturated hydrocarbon groups such as a norbornenyl group, a tricyclodecenyl group, and a tetracyclododecenyl group.

Examples of the monovalent aromatic hydrocarbon group having 6 to 20 carbon atoms include: aryl groups such as a phenyl group, a tolyl group, a xylyl group, a naphthyl group, and an anthryl group; and aralkyl groups such as a benzyl group, a phenethyl group, a naphthylmethyl group, and an anthrylmethyl group.

The saturated alicyclic ring having 3 to 20 ring atoms, which may be represented by R^{Y} and R^{Z} taken together, together with the carbon atom to which R^{Y} and R^{Z} bond, and the saturated alicyclic ring having 3 to 20 ring atoms, which may be represented by R^{U} and R^{V} taken together, together with the carbon atom to which R^{U} and RV bond are exemplified by: monocyclic saturated alicyclic rings such as a cyclopropane ring, a cyclobutane ring, a cyclopentane ring, and a cyclohexane ring; and polycyclic saturated alicyclic rings such as a norbornane ring, an adamantane ring, a tricyclodecane ring, and a tetracyclododecane ring.

The divalent hydrocarbon group having 1 to 20 carbon atoms and represented by R^{D} is exemplified by a group obtained by removing one hydrogen atom from a group exemplified for the above-described monovalent hydrocarbon group having 1 to 20 carbon atoms.

Examples of the unsaturated alicyclic ring having 4 to 20 ring atoms and represented by R^{D} and the three carbon atoms to which R^{A}, R^{B}, and R^{C} bond, respectively, include: monocyclic unsaturated alicyclic structures such as a cyclobutene structure, a cyclopentene structure, and a cyclohexene structure; polycyclic unsaturated alicyclic structures such as a norbornene structure; and the like.

Examples of the oxygen atom-containing aliphatic heterocyclic ring having 4 to 20 ring atoms, which may be represented by R^{V} and R^{W} taken together, together with the carbon atom to which R^{V} bonds, and with the oxygen atom to which R^{W} bonds include an oxacyclobutane ring, an oxacyclopentane ring, an oxacyclohexane ring, an oxacyclobutene ring, an oxacyclopentene ring, an oxacyclohexene ring, and the like.

In the case in which R^{Y} and R^{Z} each represent a monovalent hydrocarbon group having 1 to 20 carbon atoms, R^{Y} and R^{Z} each represent preferably a chain hydrocarbon group, which is preferably an alkyl group, and is more preferably a methyl group. In this case, R^{X} represents: preferably a substituted or unsubstituted chain hydrocarbon group, or a substituted or unsubstituted aromatic hydrocarbon group; more preferably an unsubstituted alkyl group, or a substituted or unsubstituted aryl group; and still more preferably a methyl group or an iodophenyl group. It is preferred that R^{X} represents an aryl group having an iodine atom bonded to the aryl group because more improving the CDU of the radiation-sensitive composition tends to be enabled.

In the case in which R^{Y} and R^{Z} taken together represent a saturated alicyclic ring having 3 to 20 ring atoms, together with the carbon atom to which R^{Y} and R^{Z} bond, the saturated alicyclic ring is preferably a monocyclic saturated alicyclic ring, and more preferably a cyclopentane ring. In this case, R^{X} represents: preferably a substituted or unsubstituted chain hydrocarbon group, or a substituted or unsubstituted aromatic hydrocarbon group; more preferably an unsubstituted alkyl group, or an unsubstituted aryl group; and still more preferably a methyl group, an ethyl group, or a phenyl group.

R^{B} represents preferably a hydrogen atom.

R^{C} represents preferably a chain hydrocarbon group, more preferably an alkyl group, and still more preferably a methyl group.

The unsaturated alicyclic ring having 4 to 20 ring atoms and represented by R^{D} together with the three carbon atoms to which R^{A}, R^{B}, and R^{C} bond, respectively is preferably a monocyclic unsaturated alicyclic ring, and more preferably a cyclohexene ring.

The acid-labile group is preferably an acid-labile group (a-1) or (a-2).

The acid-labile group (a-1) is exemplified by groups represented by the following formulae (a-1-1) to (a-1-13). The acid-labile group (a-2) is exemplified by a group represented by the following formula (a-2-1).

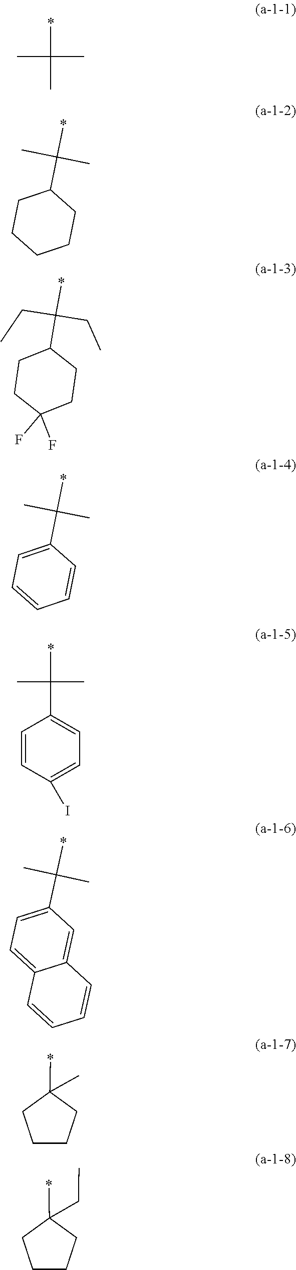

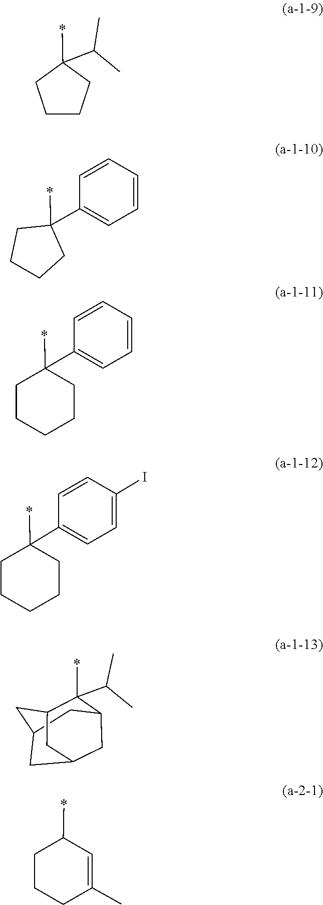

In the above formulae (a-1-1) to (a-1-13) and (a-2-1), * is as defined in the above formulae (a-1) and (a-2).

The structural unit (I) is exemplified by a structural unit represented by the following formula (I).

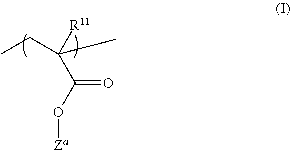

In the above formula (I), Z^{a} represents the above-described acid-labile group; and R¹¹ represents a hydrogen atom, a fluorine atom, a methyl group, or a trifluoromethyl group.

R¹¹ represents, in light of a degree of copolymerization of a monomer that gives the structural unit (I), preferably a hydrogen atom or a methyl group.

The lower limit of a proportion of the structural unit (I) included in the polymer (A) with respect to total structural units constituting the polymer (A) is preferably 20 mol %, more preferably 30 mol %, still more preferably 35 mol %, and particularly preferably 40 mol %. The upper limit of the proportion is preferably 80 mol %, more preferably 70 mol %, still more preferably 65 mol %, and particularly preferably 60 mol %. In particular, it is preferred that the proportion of the structural unit (I) is no less than 40 mol % and no greater than 60 mol % because more improving the CDU of the radiation-sensitive composition tends to be enabled.

### Structural Unit (II)

The structural unit (II) is a structural unit containing a phenolic hydroxyl group. The polymer (A) can contain one, or two or more types of the structural unit (II). It is to be noted that herein, a structural unit containing both an acid-labile group and a phenolic hydroxyl group is encompassed by the structural unit (II).

In the case of KrF exposure, EUV exposure, or electron beam exposure, the sensitivity of the radiation-sensitive composition to a radioactive ray can be more enhanced due to the polymer (A) having the structural unit (II). Therefore, in the case in which the polymer (A) has the structural unit (II), the radiation-sensitive composition can be suitably used as a radiation-sensitive composition for exposure to KrF, for exposure to EUV, or for exposure to an electron beam.

The structural unit (II) is exemplified by a structural unit represented by the following formula (II).

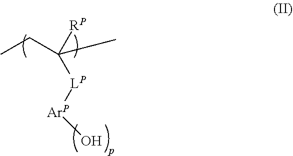

In the above formula (II), R^{P} represents a hydrogen atom, a fluorine atom, a methyl group, or a trifluoromethyl group; L^{P} represents a single bond, —COO—, —O—, or —CONH—; Ar^{P} represents a group obtained by removing (p+1) hydrogen atoms from a substituted or unsubstituted aromatic ring; and p is an integer of 1 to 3.

The term “group obtained by removing X hydrogen atom(s) from an aromatic ring” as referred to herein means a group obtained by removing X hydrogen atom(s) bonding to atom(s) constituting the aromatic ring.

R^{P} represents, in light of a degree of copolymerization of a monomer that gives the structural unit (II), preferably a hydrogen atom or a methyl group.

L^{P} represents preferably a single bond or —COO—, and more preferably a single bond.

The number of ring atoms and the type of the aromatic ring that gives Ar^{P} are exemplified by those exemplified for the aromatic ring that gives Ar¹ in the formula (1) described later. The aromatic ring that gives Ar^{P} is preferably an aromatic hydrocarbon ring having 6 to 30 ring atoms, and more preferably a benzene ring.

The aromatic ring that gives Arp may further have at least one substituent bonded to the aromatic ring. The substituent is exemplified by those exemplified as the substituent that may be incorporated in the aromatic ring that gives Ar¹ in the formula (1) described later.
p is preferably 1 or 2.

The structural unit (II) is exemplified by structural units represented by the following formulae (11-1) to (11-19).

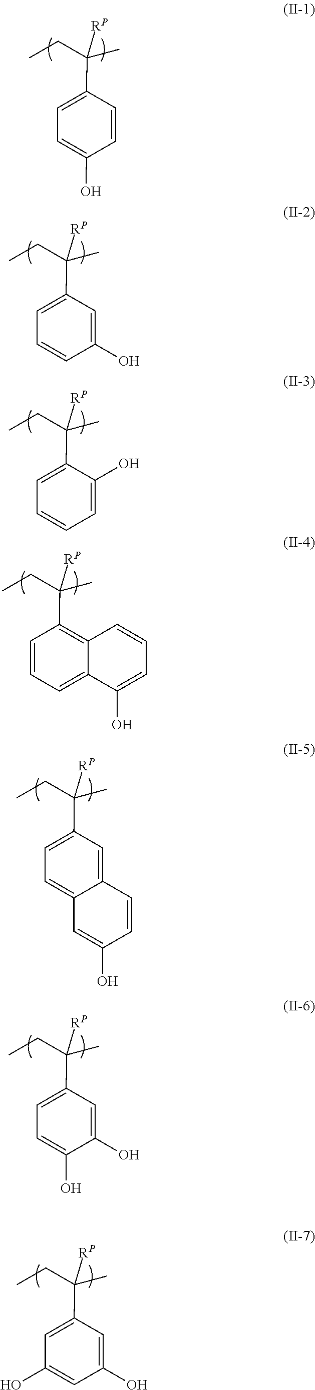

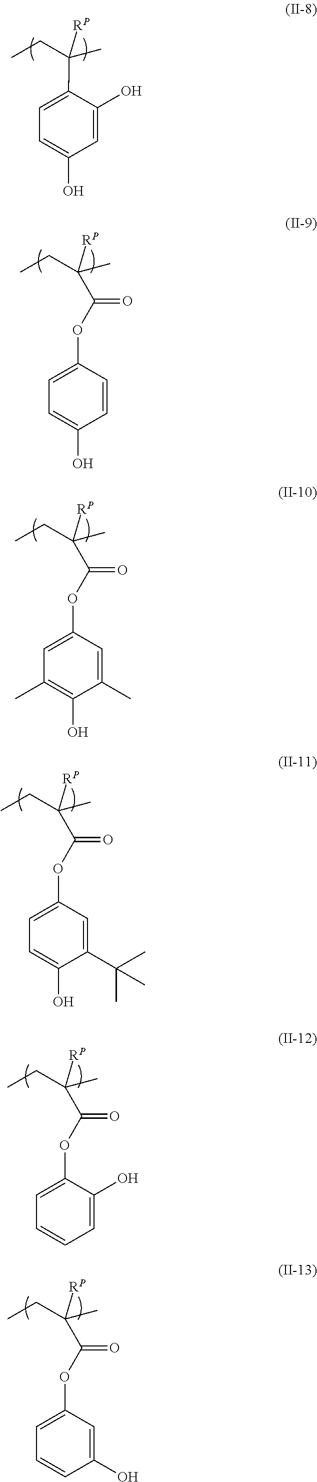

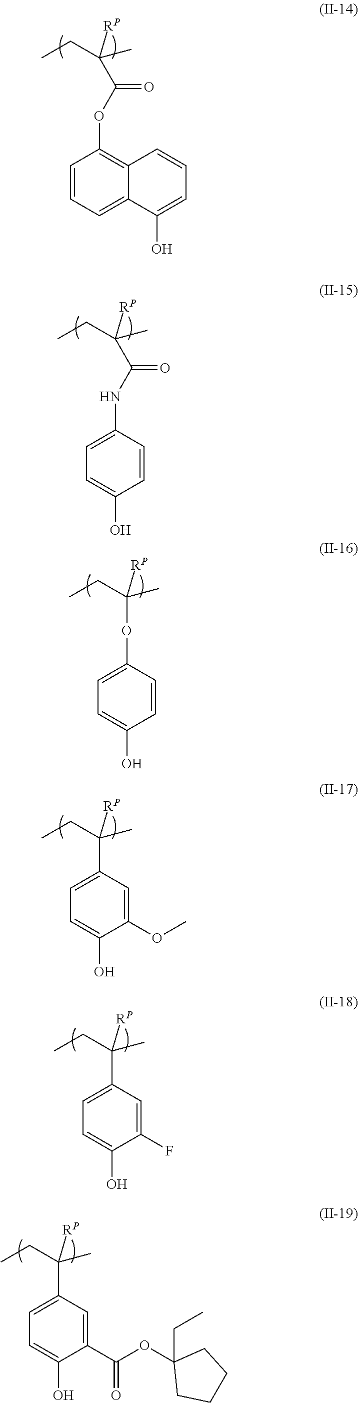

In the above formulae (II-1) to (II-19), R^{P} is as defined in the above formula (II).

In the case in which the polymer (A) has the structural unit (II), the lower limit of a proportion of the structural unit (II) included in the polymer (A) with respect to total structural units constituting the polymer (A) is preferably 20 mol %, more preferably 30 mol %, still more preferably 35 mol %, and particularly preferably 40 mol %. The upper limit of the proportion is preferably 80 mol %, more preferably 70 mol %, still more preferably 65 mol %, and particularly preferably 60 mol %. In particular, it is preferred that the proportion of the structural unit (II) is no less than 40 mol % and no greater than 60 mol % because more improving the CDU of the radiation-sensitive composition tends to be enabled.

### Other Structural Unit(s)

The other structural unit(s) is/are structural unit(s) other than the structural units (I) and (II). The other structural unit(s) is/are exemplified by: a structural unit (hereinafter, may be also referred to as “structural unit (III)”) containing a lactone structure, a cyclic carbonate structure, a sultone structure, or a combination of the same; a structural unit (hereinafter, may be also referred to as “structural unit (IV)”) containing an alcoholic hydroxyl group; and a structural unit (hereinafter, may be also referred to as “structural unit (V)”) containing a radiation-sensitive onium cation and an organic acid anion.

There may be a case in which a structural unit contained in the polymer (A) can be considered to fall under two or more categories of the structural units in an overlapping manner. For example, a structural unit which can be considered to fall under not only the structural unit (III), but also a structural unit other than the structural unit (III) may be contained. As a specific example, a structural unit represented by the following formula is considered not only to be a “structural unit containing a lactone structure” and fall under the structural unit (III), but also to be a “structural unit containing an alcoholic hydroxyl group” and fall under the structural unit (IV). In the present specification, such a structural unit is considered to be the structural unit having the lowest parenthesized number. In other words, although a structural unit represented by the following formula is a structural unit having an alcoholic hydroxyl group, the structural unit is considered to be a “structural unit containing a lactone structure” and fall under the structural unit (III).

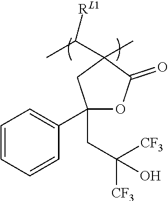

### Structural Unit (III)

The structural unit (III) is a structural unit containing a lactone structure, a cyclic carbonate structure, a sultone structure, or a combination of the same. When the polymer (A) further has the structural unit (III), adhesiveness to a substrate can be improved. The polymer (A) can contain one, or two or more types of the structural unit (III).

The structural unit (III) is exemplified by: structural units represented by the following formulae; and the like.

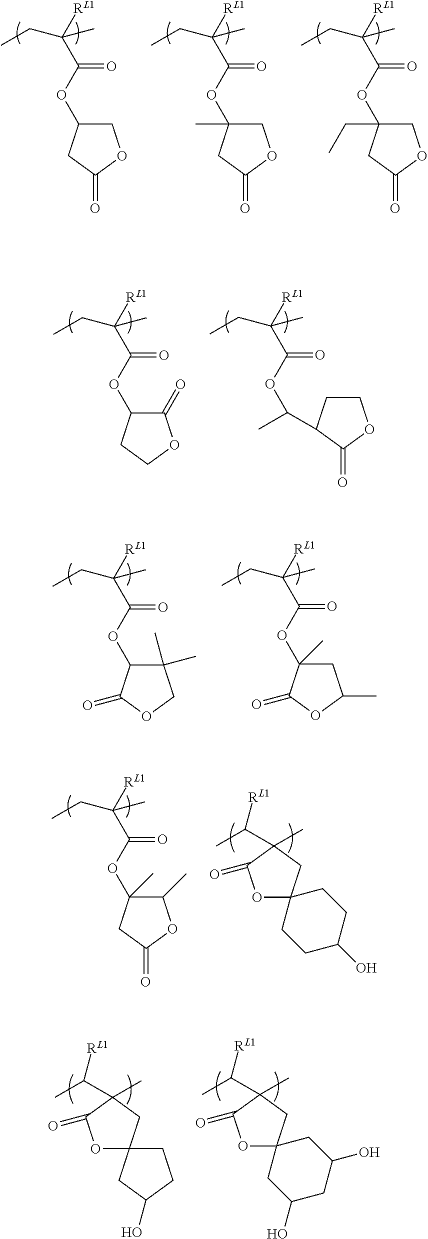

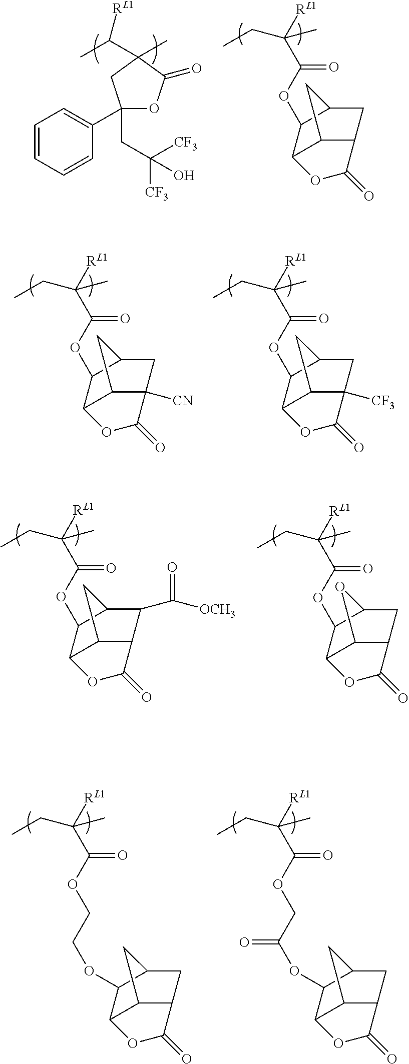

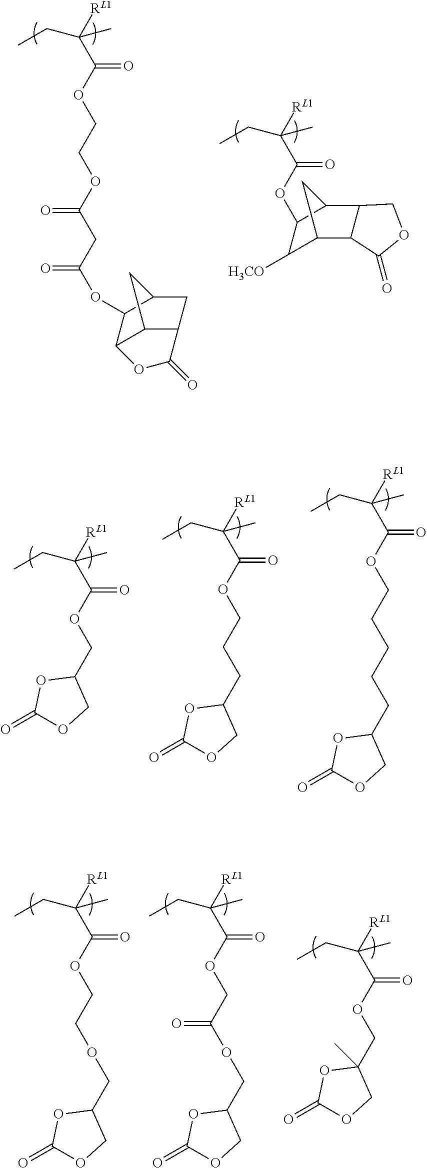

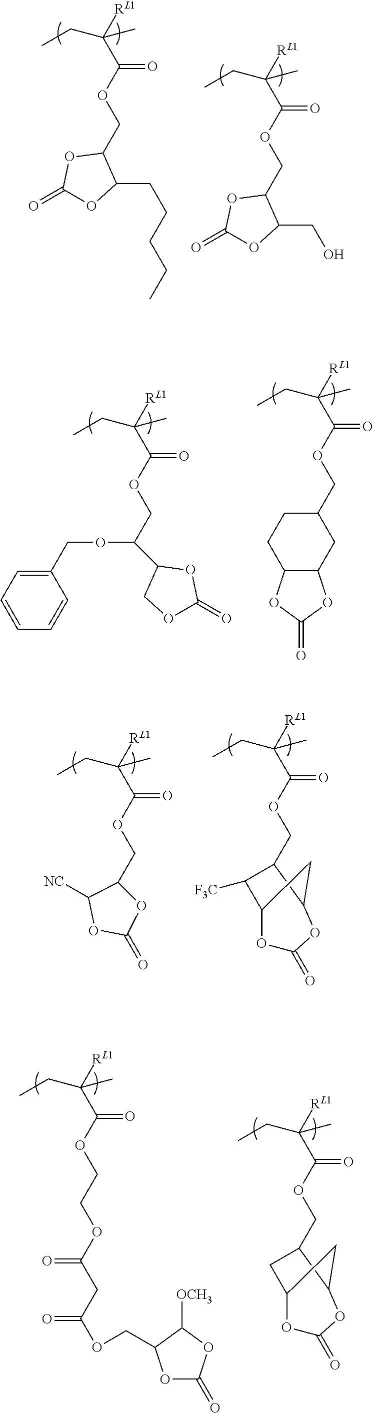

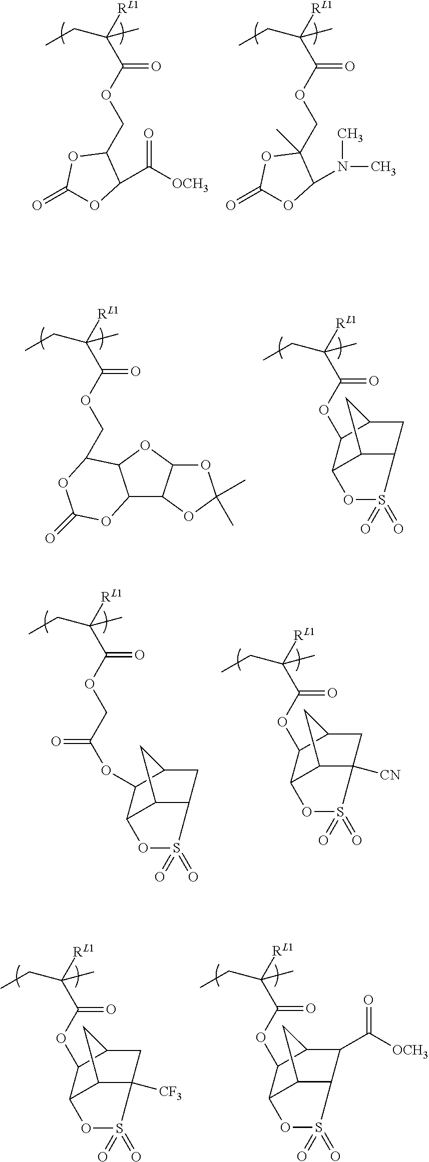

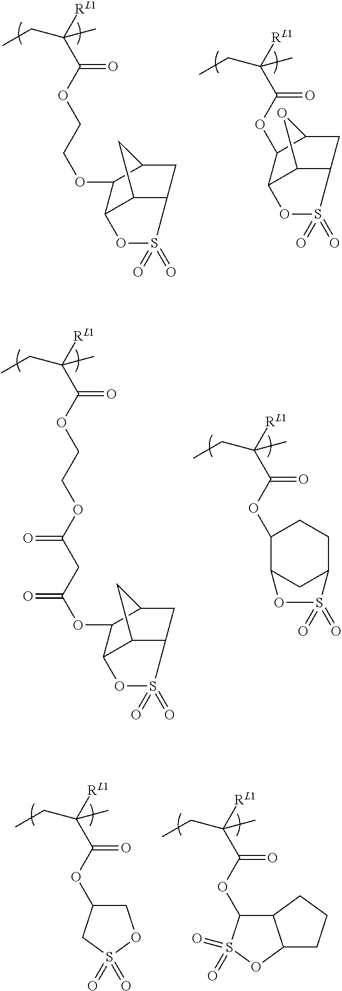

In the above formulae, R^{L1} represents a hydrogen atom, a fluorine atom, a methyl group, or a trifluoromethyl group.

The structural unit (III) is preferably a structural unit containing a lactone structure.

In the case in which the polymer (A) has the structural unit (III), the lower limit of a proportion of the structural unit (III) with respect to total structural units that constitute the polymer (A) is preferably 5 mol %, and more preferably 10 mol %. The upper limit of the proportion is preferably 40 mol %, and more preferably 30 mol %.

### Structural Unit (IV)

The structural unit (IV) is a structural unit containing an alcoholic hydroxyl group. When the polymer (A) further has the structural unit (IV), the solubility in a developer solution can be more appropriately adjusted. The polymer (A) can contain one, or two or more types of the structural unit (IV).

The structural unit (IV) is exemplified by structural units represented by the following formulae.

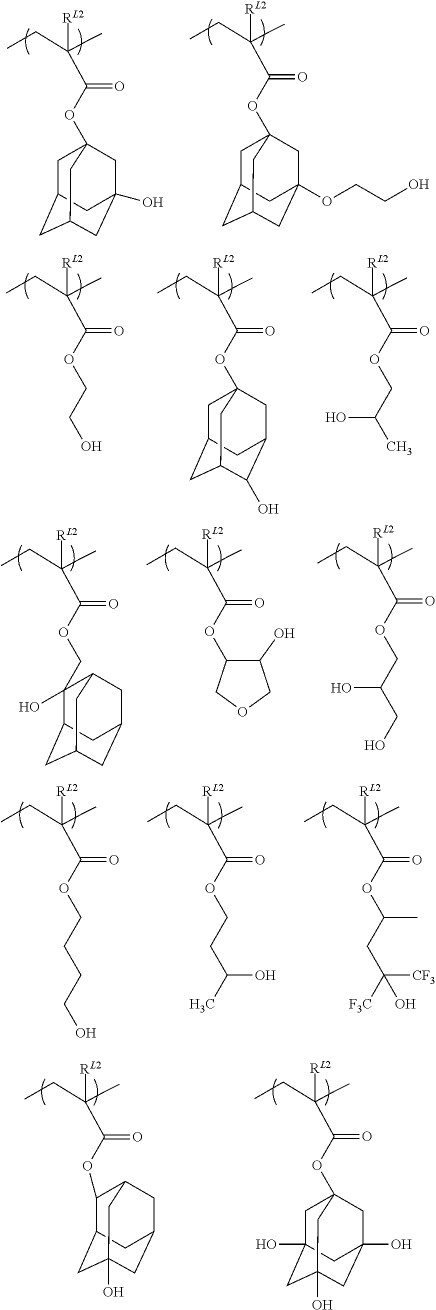

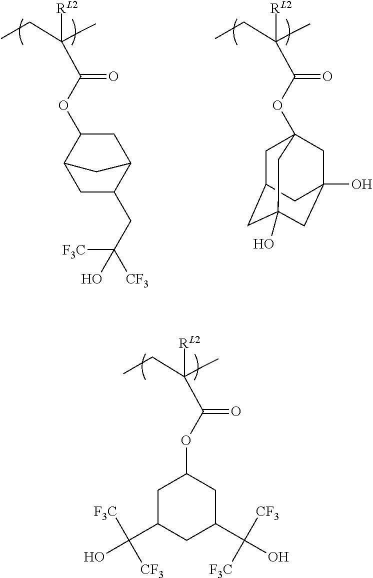

In the above formulae, R^{L2} represents a hydrogen atom, a fluorine atom, a methyl group, or a trifluoromethyl group.

In the case in which the polymer (A) has the structural unit (IV), the lower limit of a proportion of the structural unit (IV) with respect to total structural units constituting the polymer (A) is preferably 5 mol %, and more preferably 10 mol %. The upper limit of the proportion is preferably 40 mol %, and more preferably 30 mol %.

### Structural Unit (V)

The structural unit (V) is a structural unit containing a radiation-sensitive onium cation and an organic acid anion. When the polymer (A) further has the structural unit (V), the CDU of the radiation-sensitive composition can be more improved. The polymer (A) can contain one, or two or more types of the structural unit (V).

The radiation-sensitive onium cation in the structural unit (V) is exemplified by those similar to the cation (Y) described later. The organic acid anion in the structural unit (V) is exemplified by a sulfonic acid anion and a carboxylic acid anion.

In the case in which the polymer (A) has the structural unit (V), the lower limit of a proportion of the structural unit (V) with respect to total structural units constituting the polymer (A) is preferably 5 mol %, and more preferably 10 mol %. The upper limit of the proportion is preferably 30 mol %, and more preferably 20 mol %.

### (X) Anion

The anion (X) is an anion represented by the following formula (1).

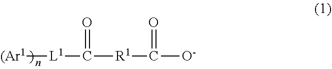

In the above formula (1), Ar¹ represents a group obtained by removing one hydrogen atom from a substituted or unsubstituted aromatic ring; n is an integer of 1 to 3, wherein in a case in which n is no less than 2, a plurality of Aris are identical or different; R¹ represents a single bond or a substituted or unsubstituted divalent hydrocarbon group; L¹ represents —O—, (*)ₙ—R²—O—, or —NR³—, wherein in a case in which n is no less than 2, L¹ represents (*)ₙ—R²—O—; * denotes a site bonding to Ar¹; R² represents a substituted or unsubstituted hydrocarbon group having a valency of (n+1); and R³ represents a hydrogen atom or a monovalent hydrocarbon group.

The number of ring atoms of the aromatic ring that gives Ar¹ is not particularly limited and is, for example, 5 to 30, or 6 to 30, and preferably 5 to 20, or 6 to 20.

The aromatic ring that gives Ar¹ is exemplified by an aromatic hydrocarbon ring having 6 to 30 ring atoms, and an aromatic heterocyclic ring having 5 to 30 ring atoms.

Examples of the aromatic hydrocarbon ring having 6 to 30 ring atoms include: a benzene ring; condensed polycyclic aromatic hydrocarbon rings such as a naphthalene ring, an anthracene ring, a fluorene ring, a biphenylene ring, a phenanthrene ring, and a pyrene ring; and ring-assembled aromatic hydrocarbon rings such as a biphenyl ring, a terphenyl ring, a binaphthalene ring, and a phenylnaphthalene ring.

Examples of the aromatic heterocyclic ring structure having 5 to 30 ring atoms include: oxygen atom-containing aromatic heterocyclic rings such as a furan ring, a pyran ring, a benzofuran ring, a benzopyran ring, and a benzodioxolane ring; nitrogen atom-containing aromatic heterocyclic rings such as a pyrrole ring, a pyridine ring, a pyrimidine ring, an indole ring, and a quinoline ring; and sulfur atom-containing aromatic heterocyclic rings such as a thiophene ring and a dibenzothiophene ring.

The aromatic ring that gives Ar¹ is preferably the aromatic hydrocarbon ring having 6 to 30 ring atoms, or the oxygen atom-containing aromatic heterocyclic ring having 5 to 30 ring atoms, and more preferably a benzene ring or a benzodioxolane ring.

It is to be noted that the aromatic ring that gives Ar¹ involves not only a case of including one above-described aromatic ring, but also a case in which two or more aromatic rings are included, and the aromatic rings are bonded to each other via a linking group.

The “linking group” as referred to herein means a group that links two or more structures. The linking group remains in the structure of a compound as a consequence of, e.g., a synthesis material or a synthesis procedure, and does not influence the effects of the present invention or has an extremely small influence on the effects of the present invention. It is to be noted that this contemplation does not suggest that all the structures aside from the linking group contribute to an exhibition of the effects of the present invention. The linking group is not particularly limited as long as it is a group that links two or more structures. The linking group is exemplified by: a carbonyl group; an ether group; a carbonyloxy group; a sulfide group; an alkanediyl group having 1 to 10 carbon atoms; or a group obtained by combining the same.

The aromatic ring that gives the aromatic ring Ar¹ may have at least one substituent bonded to the aromatic ring. The substituent is exemplified by: halogen atoms such as a fluorine atom and an iodine atom; a hydroxy group; a carboxy group; a cyano group; a nitro group; an alkyl group; a fluorinated alkyl group (a group obtained by substituting a fluorine atom for at least one hydrogen atom included in an alkyl group); an alkoxy group; an alkoxycarbonyl group; an alkoxycarbonyloxy group; an acyl group; an acyloxy group; and an oxo group (═O).

It is preferred that the aromatic ring that gives Ar¹ has at least one iodine atom bonded to the aromatic ring because more improving the sensitivity of the radiation-sensitive composition tends to be enabled. In other words, it is preferred that at least one hydrogen atom bonding to an atom constituting the aromatic ring that gives Ar¹ has been substituted with an iodine atom. The number of the substitution(s) with iodine atom(s) is not particularly limited as long as it is no less than 1. The number can be appropriately selected, and is, e.g., 1 to 5, and preferably 1 to 3.
n is preferably 1 or 2, and more preferably 1.

The divalent hydrocarbon group which may be represented by R¹ is exemplified by a group obtained by removing one hydrogen atom from a group exemplified as the above-described monovalent hydrocarbon group having 1 to 20 carbon atoms.
R¹ represents preferably a single bond.
L¹ represents preferably —O— or (*)ₙ—R²—O—, more preferably (*)ₙ—R²—O—, and still more preferably *—R²—O— (corresponding to the case in which n is 1). A case in which L₁ represents (*)ₙ—R²—O— is preferred because the sensitivity and the CDU tend to be more superior, as compared with the case in which L¹ represents —O—.

The hydrocarbon group that has a valency of (n+1) and is represented by R² is exemplified by a group obtained by removing n hydrogen atom(s) from a group exemplified as the above-described monovalent hydrocarbon group having 1 to 20 carbon atoms. R² represents preferably a chain hydrocarbon group having a valency of (n+1), and the number of the carbon atoms is preferably 1 to 20, more preferably 1 to 10, and still more preferably 1 to 5.

The hydrocarbon group which may be represented by R¹ and R² may further have at least one substituent bonded to the hydrocarbon group. The substituent is exemplified by: halogen atoms such as a fluorine atom and an iodine atom; a hydroxy group; a carboxy group; a cyano group; a nitro group; an alkoxy group; an alkoxycarbonyl group; an alkoxycarbonyloxy group; an acyl group; and an acyloxy group.

The monovalent hydrocarbon group which may be represented by R³ is exemplified by the groups exemplified as the above-described monovalent hydrocarbon group having 1 to 20 carbon atoms.
R³ represents preferably a hydrogen atom.

The anion (X) is exemplified by anions represented by the following formulae (X-1) to (X-14).

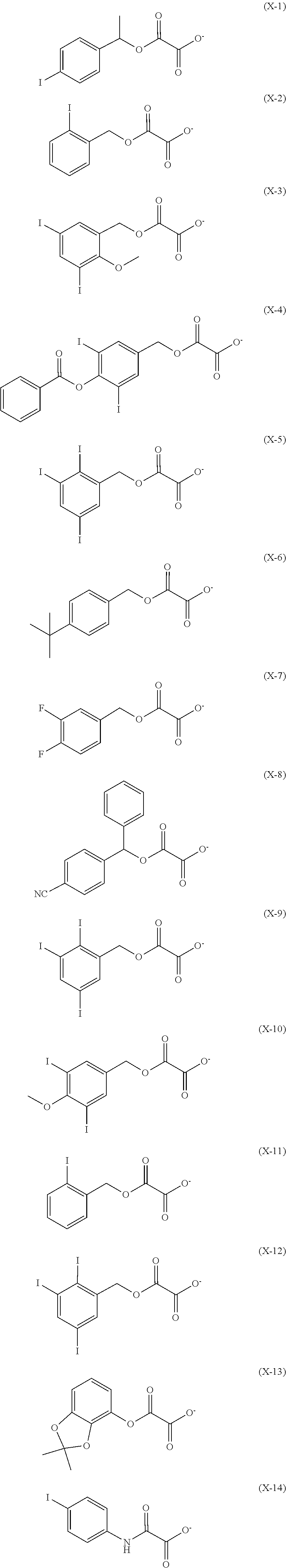

In the radiation-sensitive composition, the anion (X) may be present in the form of an anion, or in the form of a compound ionically bonding to a cation. In other words, the sentence “the radiation-sensitive composition contains the anion (X)” as referred to herein means that the radiation-sensitive composition contains the anion (X) regardless of the existing form of the anion (X).

The anion (X) is derived from a compound (hereinafter, may be also referred to as “compound (α)”) containing the anion (X). The compound (α) is exemplified by a compound containing the anion (X) and a cation. This cation is not particularly limited and may be any cation. The valency of the cation is not particularly limited, and the cation may be, for example, monovalent or divalent. In the case in which the cation is divalent or higher, the number of the anion (X) included in the compound (α) corresponds to the valency of the cation because the anion (X) is a monovalent anion.

The above cation is preferably a monovalent cation. Furthermore, the cation is classified into a radiation-sensitive onium cation and a cation other than the radiation-sensitive onium cation. Of these, a monovalent radiation-sensitive onium cation is preferred because the CDU tends to more improve.

The monovalent radiation-sensitive onium cation is not particularly limited, and any radiation-sensitive onium cation conventionally used in the technical field to which the present invention pertains can be used. Examples of the monovalent radiation-sensitive onium cation include a triphenylsulfonium cation, a diphenyliodonium cation, and the like. Alternatively, it may be a cation (Y) described later (corresponding to a monovalent radiation-sensitive onium cation).

A case in which the cation is the cation (Y) described later is preferred because the sensitivity of the radiation-sensitive composition tends to more improve, as compared with a case in which the cation is a radiation-sensitive onium cation other than the cation (Y).

In the case in which the compound (α) has a radiation-sensitive onium cation as the cation, the compound (α) typically serves as an acid diffusion control agent in the radiation-sensitive composition.

Examples of the cation other than the radiation-sensitive onium cation include a proton (H⁺), a sodium ion, a potassium ion, an ammonium ion, an oxonium ion, and a phosphonium ion.

In the case in which the compound (α) has, as the cation, the cation other than the radiation-sensitive onium cation, the compound (α) is used as an additive for the radiation-sensitive composition.

The content of the compound (α) in the radiation-sensitive composition can be appropriately predetermined depending on the purpose of blend of the compound (α). For example, in the case in which the compound (α) is used as an acid diffusion control agent, the content thereof may be the content described in the section of (C) Acid Diffusion Control Agent described later.

### (Y) Cation

The cation (Y) is a radiation-sensitive onium cation containing an aromatic ring (hereinafter, may be also referred to as “aromatic ring (y1)”) having at least one fluorine atom or fluorine atom-containing group bonded to the aromatic ring. The valency of the cation (Y) is not particularly limited, and the cation (Y) may be, for example, monovalent or divalent.

The aromatic ring that gives the aromatic ring (y1) has at least one fluorine atom or fluorine atom-containing group bonded to the aromatic ring. In other words, at least one hydrogen atom bonding to an atom constituting the aromatic ring that gives the aromatic ring (y1) has been substituted with a fluorine atom or fluorine atom-containing group. The number of the substitution(s) with fluorine atom(s) or fluorine atom-containing group(s) is not particularly limited as long as it is no less than 1. The number is, e.g., 1 to 10. A case in which the total of the fluorine atom(s) bonding to the aromatic ring (y1) and the fluorine atom(s) in the fluorine atom-containing group bonding to the aromatic ring (y1) in the entire cation (Y) is no less than 4 is preferred because the sensitivity and/or the CDU of the radiation-sensitive composition tends to more improve, as compared with a case in which the total of the fluorine atoms is no greater than 3.

The number of ring atoms and the type of the aromatic ring that gives the aromatic ring (y1) are exemplified by those exemplified for the aromatic ring that gives Ar¹ in the above formula (1). The aromatic ring that gives the aromatic ring (y1) is preferably an aromatic hydrocarbon ring having 6 to 30 ring atoms or an aromatic heterocyclic ring having 5 to 30 ring atoms, more preferably a benzene ring, a condensed polycyclic aromatic hydrocarbon ring, or a sulfur atom-containing polycyclic aromatic heterocyclic ring, and still more preferably a benzene ring, a naphthalene ring, or a dibenzothiophene ring.

The “fluorine atom-containing group” as referred to herein means a group having at least one fluorine atom. The fluorine atom-containing group is exemplified by fluorinated hydrocarbon groups (groups obtained by substituting a fluorine atom for at least one hydrogen atom included in a hydrocarbon group). The fluorine atom-containing group is preferably a fluorinated alkyl group, and more preferably a trifluoromethyl group.

The aromatic ring that gives the aromatic ring (y1) may further have at least one substituent, other than the fluorine atom and the fluorine atom-containing group, bonded to the aromatic ring. The substituent is exemplified by groups exemplified as a substituent that may be contained in the aromatic ring that gives Ar¹ in the formula (1) described later, except for a fluorine atom and fluorine atom-containing groups.

The cation (Y) is exemplified by a monovalent cation represented by the following formula (r-a).

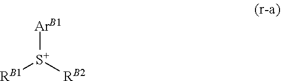

In the above formula (r-a), Ar^{B1} represents a group obtained by removing one hydrogen atom from an aromatic ring having at least one fluorine atom or fluorine atom-containing group bonded to the aromatic ring; and R^{B1} and R^{B2} each independently represent a group obtained by removing one hydrogen atom from a substituted or unsubstituted aromatic hydrocarbon ring having 6 to 20 ring atoms, or R^{B1} and R^{B2} taken together represent a substituted or unsubstituted polycyclic sulfur atom-containing aromatic heterocyclic ring having 9 to 30 ring atoms, together with the sulfur atom to which R^{B1} and R^{B2} bond.

The aromatic ring that gives Ar^{B1} is described as the aromatic ring (y1). The aromatic hydrocarbon ring that has 6 to 20 ring atoms, which may be represented by R^{B1} or R^{B2} is described as a preferred mode of the above-described aromatic ring that gives the aromatic ring (y1). The polycyclic sulfur atom-containing aromatic heterocyclic ring having 9 to 30 ring atoms, which may be represented by R^{B1} and R^{B2} taken together, together with the sulfur atom to which R^{B1} and R^{B2} bond is also described as a preferred mode of the above aromatic ring (y1).

The cation (Y) is exemplified by monovalent cations represented by the following formulae (r-a-1) to (r-a-7).

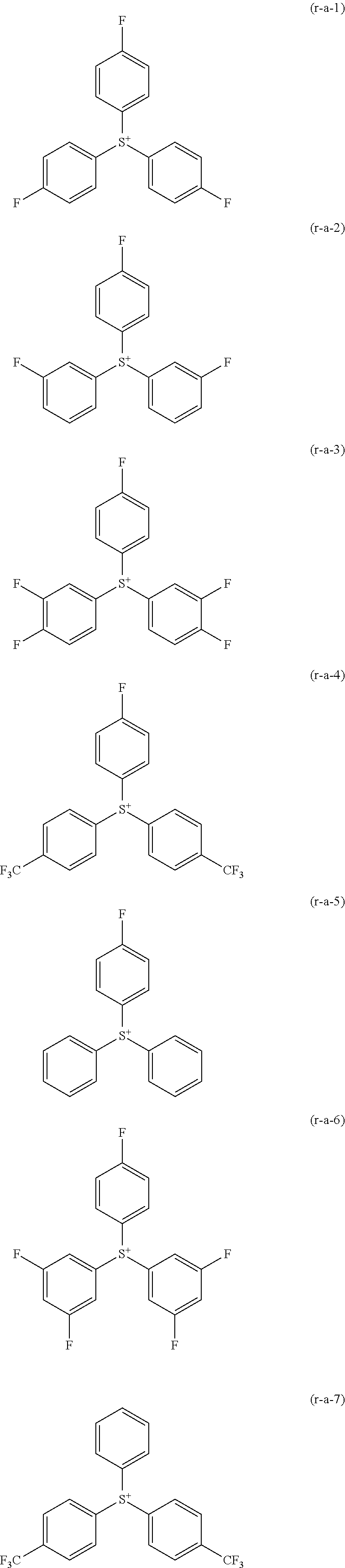

In the radiation-sensitive composition, the cation (Y) may be present in the form of a cation, or in the form of a compound (onium salt) ionically bonding to an anion. In other words, the sentence “the radiation-sensitive composition contains the cation (Y)” as referred to herein means that the radiation-sensitive composition contains the cation (Y) regardless of the existing form of the cation (Y).

The cation (Y) is derived from a compound (hereinafter, may be also referred to as “compound (β)”) containing the cation (Y). The compound (β) is exemplified by a compound having the cation (Y) and an anion. The anion is not particularly limited, and may be any anion. The valency of the anion is not particularly limited, and the anion may be, for example, monovalent or divalent.

The anion is preferably a monovalent anion. Examples of the monovalent anion include a sulfonic acid anion, a carboxylic acid anion, an imide anion, a chloride ion, a bromide ion, and a hydroxide ion.

In the case in which the compound (β) has a sulfonic acid anion as the anion, the compound (β) typically serves as a radiation-sensitive acid generating agent in the radiation-sensitive composition.

In the case in which the compound (β) has a carboxylic acid anion as the anion, the compound (β) typically serves as an acid diffusion control agent in the radiation-sensitive composition.

The carboxylic acid anion may be the anion (X) described above. In this case, the compound (β) is the compound containing the anion (X) and the cation (Y), and conceptually also corresponds to the compound (α).

The sulfonic acid anion is exemplified by anions represented by the following formulae (p-1) to (p-9).

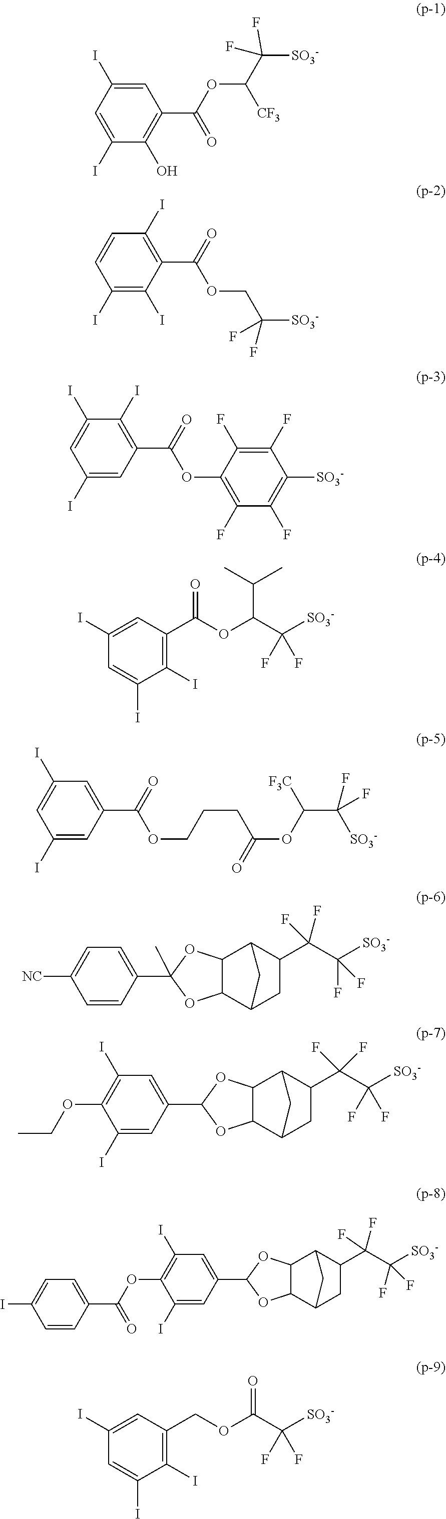

The carboxylic acid anion other than the above anion (X) is exemplified by an anion represented by the following formula (q-1) or (q-2).

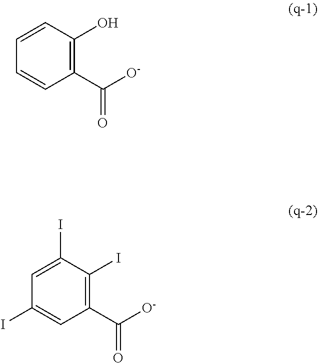 [SMILES: O=C([O-])c1ccccc1O ; O=C([O-])c1cc(I)cc(I)c1I]

The content of the compound (β) in the radiation-sensitive composition can be appropriately predetermined depending on the purpose of blend of the compound (β). For example, in the case in which the compound (β) is used as a radiation-sensitive acid generating agent, the content thereof may be the content described in the section of (B) Acid Generating Agent described later; and in the case in which the compound (β) is used as an acid diffusion control agent, the content thereof may be the content described in the section of (C) Acid Diffusion Control Agent described later.

The lower limit of a molar ratio Y/X of the content of the cation (Y) to the content of the anion (X) in the radiation-sensitive composition is preferably 0.5, more preferably 0.75, and still more preferably 1.0, and may be preferably 1.5, 2.0, 2.5, or 3.0. In the case in which the molar ratio Y/X is greater than 2.5, the sensitivity tends to more improve. The upper limit of the molar ratio Y/X is preferably 5.0, more preferably 4.5, and still more preferably 4.0.

### (B) Acid Generating Agent

The acid generating agent (B) is a compound that generates an acid upon an irradiation with a radioactive ray. The acid generated from the acid generating agent (B) upon an irradiation with a radioactive ray allows an acid-labile group included in the polymer (A) to be dissociated, thereby generating a carboxy group, a phenolic hydroxyl group, etc., whereby a difference in solubility of the resist film in the developer solution is generated between the light-exposed regions and the light-unexposed regions, and thus formation of the resist pattern is enabled. The radiation-sensitive composition can contain one, or two or more types of the acid generating agent (B).

The acid generating agent (B) may be a compound (compound (β)) containing the cation (Y), or may be a compound other than the compound (β).

The compound (β) that serves as an acid generating agent is described in the above section of (Y) Cation.

As the acid generating agent other than the compound (β), any compound can be used without any particular limitation, as long as it does not correspond to the compound (β) and can be used as a radiation-sensitive acid generating agent. The acid generating agent other than the compound (β) is exemplified by an onium salt compound obtained by appropriately combining the above-described radiation-sensitive onium cation other than the cation (Y), and an anionic moiety of a strong acid represented by each of the above formulae (p-1) to (p-9); an N-sulfonyloxyimide compound; a sulfonimide compound; a halogen-containing compound; and a diazoketone compound. Furthermore, specific examples of the acid generating agent (B) include a compound disclosed in paragraphs 0080 to 0113 of Japanese Unexamined Patent Application, Publication No. 2009-134088.

It is preferred that the radiation-sensitive composition contains a compound (compound (β)) containing the cation (Y) as the acid generating agent (B) because the sensitivity of the radiation-sensitive composition tends to more improve.

The lower limit of a content of the acid generating agent (B) in the radiation-sensitive composition, with respect to 100 parts by mass of the polymer (A), is preferably 10 parts by mass, more preferably 20 parts by mass, still more preferably 30 parts by mass, and particularly preferably 40 parts by mass. The upper limit of the content is preferably 70 parts by mass, more preferably 60 parts by mass, and still more preferably 50 parts by mass. A case in which the content of the acid generating agent (B) in the radiation-sensitive composition is greater than 40 parts by mass is preferred because the CDU of the radiation-sensitive composition tends to more improve, as compared with a case in which the content is no greater than 40 parts by mass.

### (C) Acid Diffusion Control Agent

The acid diffusion control agent (C) controls a diffusion phenomenon, in the resist film, of the acid generated, upon exposure, from the acid generating agent (B) and the like, thereby serving to control unwanted chemical reactions in the light-unexposed regions. The radiation-sensitive composition can contain one, or two or more types of the acid diffusion control agent (C).

The acid diffusion control agent (C) may be a compound (compound (α)) containing the anion (X), or may be a compound other than the compound (α).

The compound (α) that serves as an acid diffusion control agent is described in the above section of (X) Anion.

The compound other than the compound (α) is exemplified by: nitrogen atom-containing compounds other than the compound (α); and compounds (hereinafter, may be also referred to as “photodegradable base”), other than the compound (α), that generate a weak acid upon exposure.

Examples of the nitrogen atom-containing compound include: amine compounds such as tripentylamine and trioctylamine; amide group-containing compounds such as formamide and N,N-dimethylacetamide; urea compounds such as urea and 1,1-dimethylurea; nitrogen-containing heterocyclic compounds such as pyridine, N-(undecylcarbonyloxyethyl)morpholine, and N-t-pentyloxycarbonyl-4-hydroxypiperidine; and the like.

The photodegradable base is exemplified by a compound obtained by appropriately combining the above-described radiation-sensitive onium cation and an anionic moiety of a weak acid represented by the above formula (q-1) or (q-2). The photodegradable base generates a weak acid in light-exposed regions and enhances the solubility or insolubility of the polymer (A) in a developer solution, and consequently roughness of surfaces of the light-exposed regions after the development is reduced. On the other hand, the photodegradable base exerts a superior acid-capturing function due to the anion in light-unexposed regions and serves as a quencher, and thus captures the acid diffused from the light-exposed regions. In other words, since the photodegradable base serves as a quencher only at the light-unexposed regions, the contrast resulting from an elimination reaction of an acid-labile group is improved, and consequently the resolution can be improved. Due to generating an acid upon exposure, the photodegradable base may be also referred to as a radiation-sensitive acid generating agent in a broad sense; however, under conditions in which the acid generated from the acid generating agent (B) upon exposure allows for dissociation of an acid-labile group, the photodegradable base does not cause dissociation of the acid-labile group upon exposure, and thus is clearly distinguished from the radiation-sensitive acid generating agent.

It is preferred that the radiation-sensitive composition contains, as the acid diffusion control agent (C), a compound (compound (α)) containing the anion (X) because the CDU of the radiation-sensitive composition tends to more improve.

In the case in which the radiation-sensitive composition contains the acid diffusion control agent (C), the lower limit of a proportion of the acid diffusion control agent (C) in the radiation-sensitive composition, with respect to 100 mol % of the acid generating agent (B), is preferably 20 mol %, and more preferably 30 mol %. The upper limit of the proportion is preferably 200 mol %, more preferably 120 mol %, and still more preferably 80 mol %.

### (D) Organic Solvent

The radiation-sensitive composition typically contains the organic solvent (D). The organic solvent (D) is not particularly limited as long as it is a solvent capable of dissolving or dispersing at least the polymer (A), the compound containing the anion (X), and the compound containing the cation (Y), as well as the acid generating agent (B), the acid diffusion control agent (C), and the other optional component(s), which is/are contained as needed.

The organic solvent (D) is exemplified by an alcohol solvent, an ether solvent, a ketone solvent, an amide solvent, an ester solvent, and a hydrocarbon solvent. The radiation-sensitive composition can contain one, or two or more types of the organic solvent (D).

Examples of the alcohol solvent include:
aliphatic monohydric alcohol solvents such as 4-methyl-2-pentanol, n-hexanol, and diacetone alcohol;
alicyclic monohydric alcohol solvents such as cyclohexanol;
polyhydric alcohol solvents such as 1,2-propylene glycol; and
polyhydric alcohol partial ether solvents such as propylene glycol monomethyl ether.

Examples of the ether solvent include:
dialkyl ether solvents such as diethyl ether, dipropyl ether, dibutyl ether, dipentyl ether, diisoamyl ether, dihexyl ether, and diheptyl ether;
cyclic ether solvents such as tetrahydrofuran and tetrahydropyran; and aromatic ring-containing ether solvents such as diphenyl ether and anisole.

Examples of the ketone solvent include:
chain ketone solvents such as acetone, methyl ethyl ketone, methyl n-propyl ketone, methyl n-butyl ketone, diethyl ketone, methyl iso-butyl ketone, 2-heptanone, ethyl n-butyl ketone, methyl n-hexyl ketone, di-iso-butyl ketone, and trimethylnonanone;
cyclic ketone solvents such as cyclopentanone, cyclohexanone, cycloheptanone, cyclooctanone, and methylcyclohexanone; and
2,4-pentanedione, acetonylacetone, and acetophenone.

Examples of the amide solvent include:
cyclic amide solvents such as N,N′-dimethylimidazolidinone and N-methylpyrrolidone; and
chain amide solvents such as N-methylformamide, N,N-dimethylformamide, N,N-diethylformamide, acetamide, N-methylacetamide, N,N-dimethylacetamide, and N-methylpropionamide.

Examples of the ester solvent include:
monocarboxylic acid ester solvents such as n-butyl acetate, ethyl lactate, and methyl 2-hydroxyisobutyrate;
lactone solvents such as y-butyrolactone and valerolactone;
polyhydric alcohol carboxylate solvents such as propylene glycol acetate;
polyhydric alcohol partial ether carboxylate solvents such as propylene glycol monomethyl ether acetate;
polyhydric carboxylic acid diester solvents such as diethyl oxalate; and
carbonate solvents such as dimethyl carbonate and diethyl carbonate.

Examples of the hydrocarbon solvent include:
aliphatic hydrocarbon solvents such as n-pentane and n-hexane; and
aromatic hydrocarbon solvents such as toluene and xylene.

The organic solvent (D) is preferably the alcohol solvent, the ester solvent, or a combination of the same, more preferably the polyhydric alcohol partial ether solvent, the polyhydric alcohol partial ether carboxylate solvent, the monocarboxylic acid ester solvent, or a combination of the same, and still more preferably propylene glycol monomethyl ether, propylene glycol monomethyl ether acetate, methyl 2-hydroxyisobutyrate, or a combination of the same.

In the case of the radiation-sensitive composition containing the organic solvent (D), the lower limit of a proportion of the organic solvent (D) with respect to total components contained in the radiation-sensitive composition is preferably 50% by mass, more preferably 60% by mass, still more preferably 70% by mass, and particularly preferably 80% by mass. The upper limit of the proportion is preferably 99.9% by mass, more preferably 99.5% by mass, and still more preferably 99.0% by mass.

### (F) Polymer

The polymer (F) is a polymer that differs from the polymer (A), and has a percentage content of fluorine atoms which is greater than that of the polymer (A). In general, a more hydrophobic polymer than a polymer that serves as a base polymer tends to be localized in a resist film surface layer. Since the polymer (F) has a percentage content of fluorine atoms which is greater than that of the polymer (A), due to characteristics resulting from the hydrophobicity, the polymer (F) tends to be localized in the resist film surface layer. As a result, in the case in which the radiation-sensitive composition contains the polymer (F), a cross-sectional shape of a resist pattern to be formed is expected to be favorable. The radiation-sensitive composition may contain the polymer (F) as, for example, a surface conditioning agent of a resist film. The radiation-sensitive composition may contain one, or two or more types of the polymer (F).

### Other Optional Component(s)

The other optional component(s) is/are exemplified by a surfactant and the like. The radiation-sensitive composition can contain one, or two or more types of the other optional component(s).

### Method of Forming Resist Pattern

The method of forming a resist pattern includes: a step (hereinafter, may be also referred to as “applying step”) of applying a radiation-sensitive composition directly or indirectly on a substrate to form a resist film; a step (hereinafter, may be also referred to as “exposing step”) of exposing the resist film; and a step (hereinafter, may be also referred to as “developing step”) of developing the resist film exposed.

In the applying step, the above-described radiation-sensitive composition is used as the radiation-sensitive composition. Therefore, the method of forming a resist pattern enables favorably forming a resist pattern because the radiation-sensitive composition that is superior in sensitivity, CDU, and storage stability is used.

Each step included in the method of forming a resist pattern is described below.

### Applying Step

In this step, the radiation-sensitive composition is applied directly or indirectly on a substrate. By this step, a resist film is formed directly or indirectly on the substrate.

In this step, the above-described radiation-sensitive composition is used as the radiation-sensitive composition.

The substrate is exemplified by conventionally well-known substrates such as a silicon wafer, and a wafer coated with silicon dioxide or aluminum.

The application procedure is exemplified by spin coating, cast coating, roll coating, and the like. After the application, prebaking (hereinafter, may be also referred to as “PB”) may be carried out as needed for evaporating the solvent remaining in the coating film. A PB temperature and a PB time period are not particularly limited, and the PB is performed, for example, at a temperature of 60° C. or higher and 150° C. or lower for a time period of no less than 5 sec and no greater than 300 sec. An average thickness of the resist film formed is not particularly limited, and is, e.g., no less than 10 nm and no greater than 1,000 nm.

### Exposing Step

In this step, the resist film formed by the applying step is exposed. This exposure is carried out by irradiation with a radioactive ray through a photomask (as the case may be, through a liquid immersion medium such as water). As the radioactive ray, a far ultraviolet ray, EUV, or an electron beam is preferred; an ArF excimer laser beam (wavelength: 193 nm), a KrF excimer laser beam (wavelength: 248 nm), EUV (wavelength: 13.5 nm), or an electron beam is more preferred; a KrF excimer laser beam, EUV, or an electron beam is still more preferred; and EUV or an electron beam is particularly preferred.

It is preferred that post exposure baking (hereinafter, may be also referred to as “PEB”) is carried out after the exposure. This PEB enables increasing a difference in solubility in a developer solution between light-exposed regions and light-unexposed regions. A PEB temperature and a PEB time period are not particularly limited, and the PEB can be performed, for example, at a temperature of 50° C. or higher and 180° C. or lower for a time period of no less than 5 sec and no greater than 600 sec.

### Developing Step

In this step, the resist film exposed is developed. Accordingly, formation of a predetermined resist pattern is enabled. The development procedure in the developing step may be carried out by either development with an alkali, or development with an organic solvent.

In the case of the development with an alkali, the developer solution for use in the development is exemplified by: alkaline aqueous solutions prepared by dissolving at least one alkaline compound such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, ethyldimethylamine, triethanolamine, tetramethylammonium hydroxide (hereinafter, may be also referred to as “TMAH”), pyrrole, piperidine, choline, 1,8-diazabicyclo-[5.4.0]-7-undecene, and 1,5-diazabicyclo-[4.3.0]-5-nonene. Of these, an aqueous TMAH solution is preferred, and a 2.38% by mass aqueous TMAH solution is more preferred.

In the case of the development with an organic solvent, the developer solution is exemplified by the organic solvents exemplified as the organic solvent (D) in the above-described radiation-sensitive composition.

### Compound

The compound is a compound represented by the following formula (2). The compound can be suitably used as a component of the radiation-sensitive composition described above. More specifically, the compound can be suitably used as the above-described compound (compound (α)) containing the anion (X).

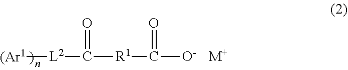

In the above formula (2), Ar¹ represents a group obtained by removing one hydrogen atom from a substituted or unsubstituted aromatic ring; n is an integer of 1 to 3, wherein in a case in which n is no less than 2, a plurality of Aris are identical or different; R¹ represents a single bond or a substituted or unsubstituted divalent hydrocarbon group; L² represents (*)ₙ—R²—O—; R² represents a substituted or unsubstituted hydrocarbon group having a valency of (n+1); * denotes a site bonding to Ar¹; and M⁺ represents a monovalent cation.

Ar¹, n, R¹, and R² are as described in the description of the above formula (1). Preferred modes thereof are also the same as the preferred modes described in the above formula (1).

The monovalent cation that gives M⁺ corresponds to the monovalent cation among the cations described in the above section of (X) Anion. The preferred modes are also the same.

### EXAMPLES

Hereinafter, the present invention is explained in detail by way of Examples, but the present invention is not in any way limited to these Examples. Measuring methods for various types of physical property values are shown below.

### Weight Average Molecular Weight (Mw), Number Average Molecular Weight (Mn), and Polydispersity Index (Mw/Mn)

Measurements of the Mw and the Mn of the polymer were carried out in accordance with the conditions described in the above section of “Method for Measuring Mw and Mn”. The polydispersity index (Mw/Mn) of the polymer was calculated from the measurement results of the Mw and the Mn.

### Synthesis of Compound (Z)

According to the following procedure, compounds (hereinafter, may be also referred to as “compounds (Z-1) to (Z-15)”) to serve as the compound (Z) and represented by the following formulae (Z-1) to (Z-15) were synthesized. The compounds (Z-1) to (Z-11) and (Z-14) to (Z-15) correspond to both the above-described compound (α) and the above-described compound (β), the compound (Z-12) corresponds to the above-described compound (α), and the compound (Z-13) corresponds to the above-described compound (β).

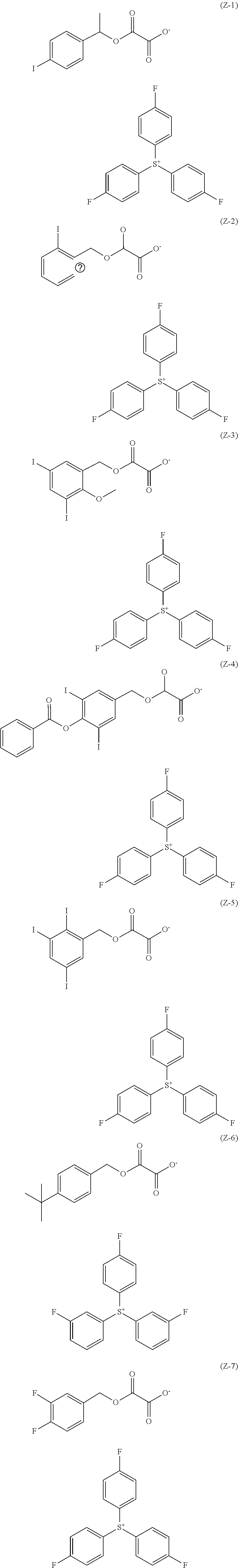

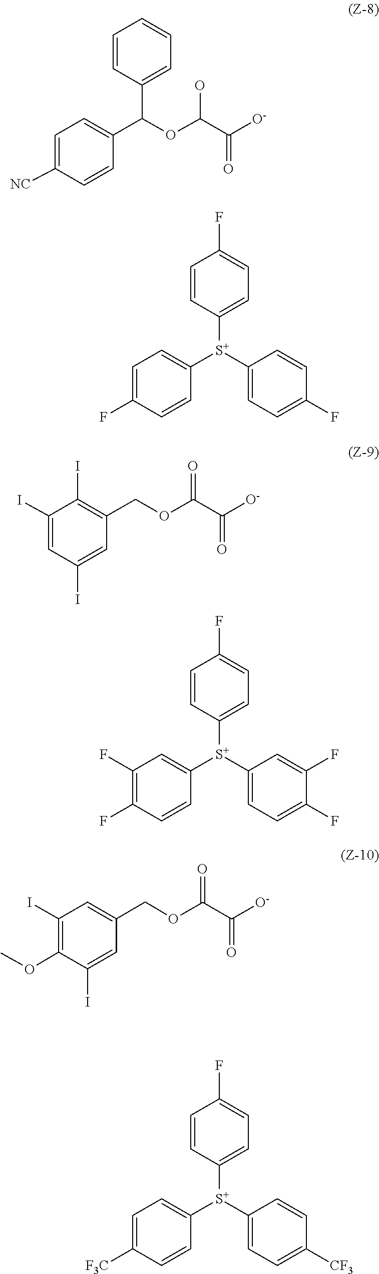 [SMILES: N#Cc1ccc(C(OC(O)C(=O)[O-])c2ccccc2)cc1 ; Fc1ccc([S+](c2ccc(F)cc2)c2ccc(F)cc2)cc1 ; O=C([O-])C(=O)OCc1cc(I)cc(I)c1I ; Fc1ccc([S+](c2ccc(F)c(F)c2)c2ccc(F)c(F)c2)cc1 ; COc1c(I)cc(COC(=O)C(=O)[O-])cc1I ; Fc1ccc([S+](c2ccc(C(F)(F)F)cc2)c2ccc(C(F)(F)F)cc2)cc1]

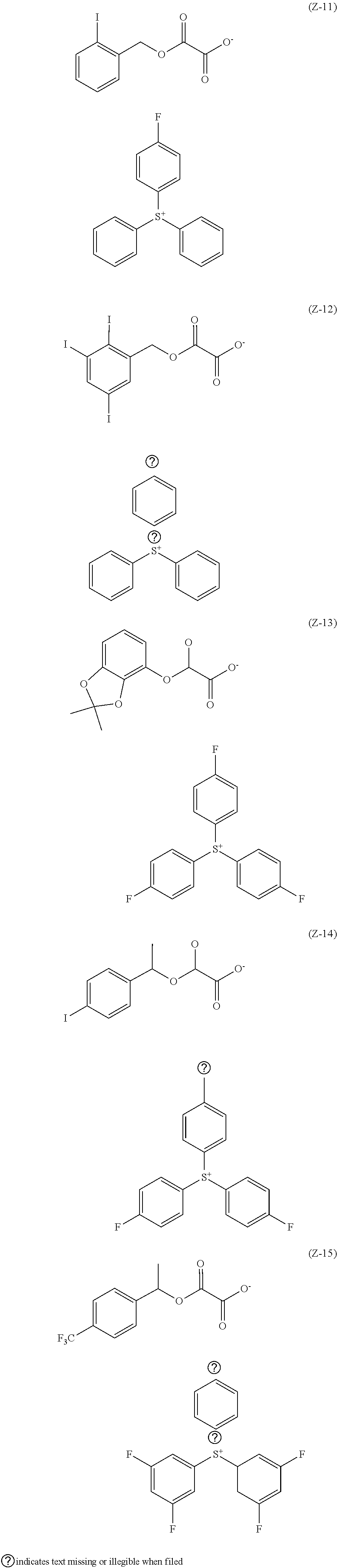

### Synthesis Example 1-1: Synthesis of Compound (Z-1)

Into a vessel containing tetrahydrofuran (100 mL) was charged a compound (P-1) (20 mmol), and a thus resulting mixture was cooled with ice. Oxalyl chloride (40 mmol) was added dropwise to the vessel, and stirring was performed at room temperature for 5 hours. After cooling with ice, 100 mL of ultra-pure water was added dropwise. Ethyl acetate was added for extraction. The organic layer was subjected to back extraction with an aqueous solution of sodium hydrogen carbonate. To the aqueous layer was added a 2 N aqueous solution of hydrochloric acid, and was added ethyl acetate for extraction. The organic layer was washed with water, dried over sodium sulfate, and filtered off. The solvent was distilled away to give a compound (PP-1).

The compound (PP-1) (10 mmol), tris(4-fluorophenyl)sulfonium bromide (10 mmol), and sodium hydrogen carbonate (20 mmol) were added to a vessel containing dichloromethane (100 mL) and ultra-pure water (100 mL). After stirring at room temperature for 5 hours, the organic layer was washed with water, and dried over sodium sulfate. After filtration, the solvent was distilled away to give a compound (Z-1).

A synthesis scheme of the compound (Z-1) is shown below.

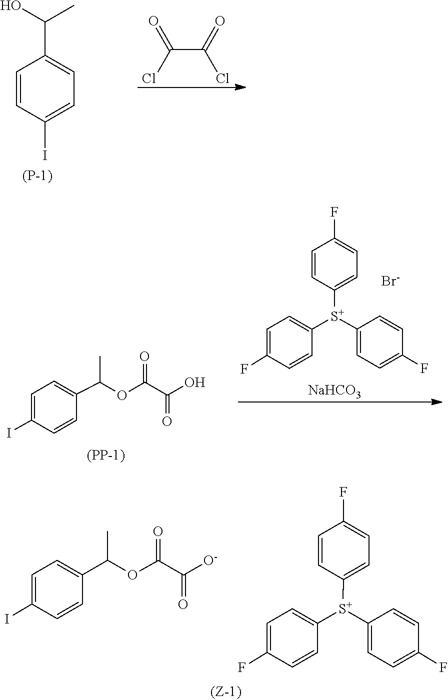

### Synthesis Examples 1-2 to 1-15: Syntheses of Compounds (Z-2) to (Z-15)

Compounds (Z-2) to (Z-15) were synthesized in a similar manner to Synthesis Example 1-1 except that the precursor was appropriately changed.

### Synthesis of Polymer (A)

According to the following procedure, polymers (A-1) to (A-19) to serve as the polymer (A) were synthesized. For synthesis of the polymer (A), compounds (hereinafter, may be also referred to as “monomers (M-1) to (M-19) represented by the following formulae (M-1) to (M-19) were used. In the following Synthesis Examples, unless otherwise specified particularly, the term “parts by mass” means a value, provided that the total mass of the monomers used was 100 parts by mass, and the term “mol %” means a value, provided that the total mol number of the monomers used accounted for 100 mol %.

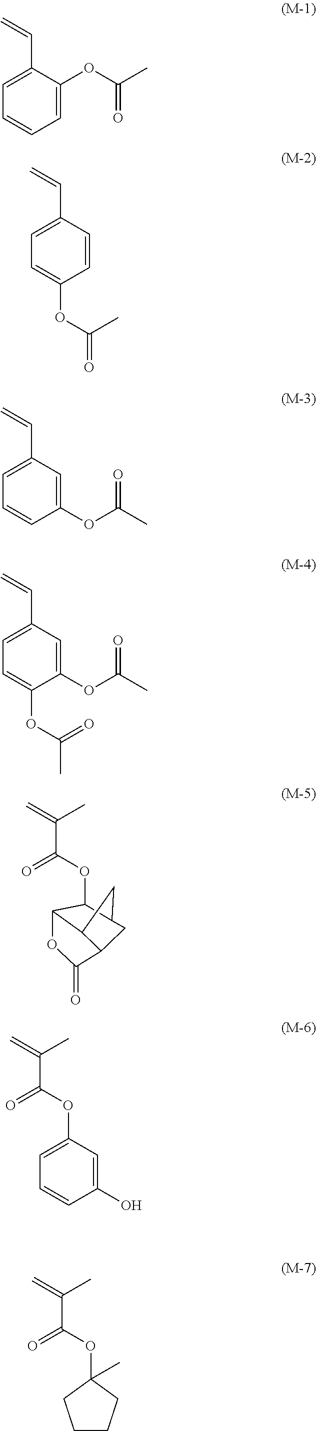

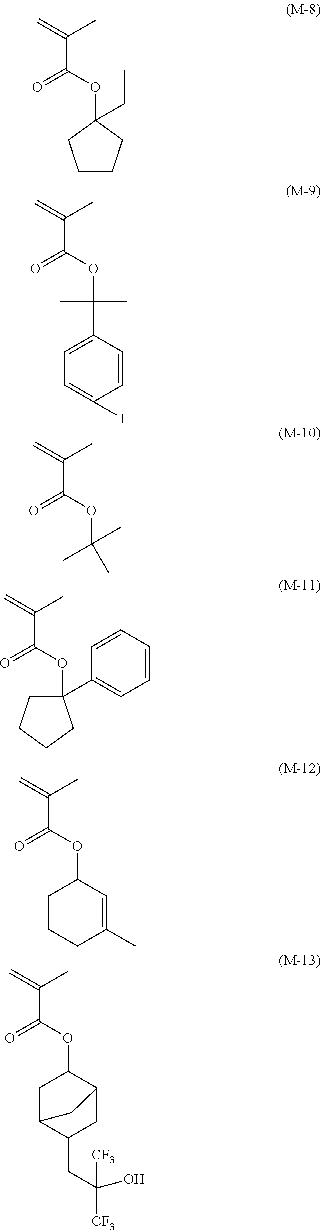

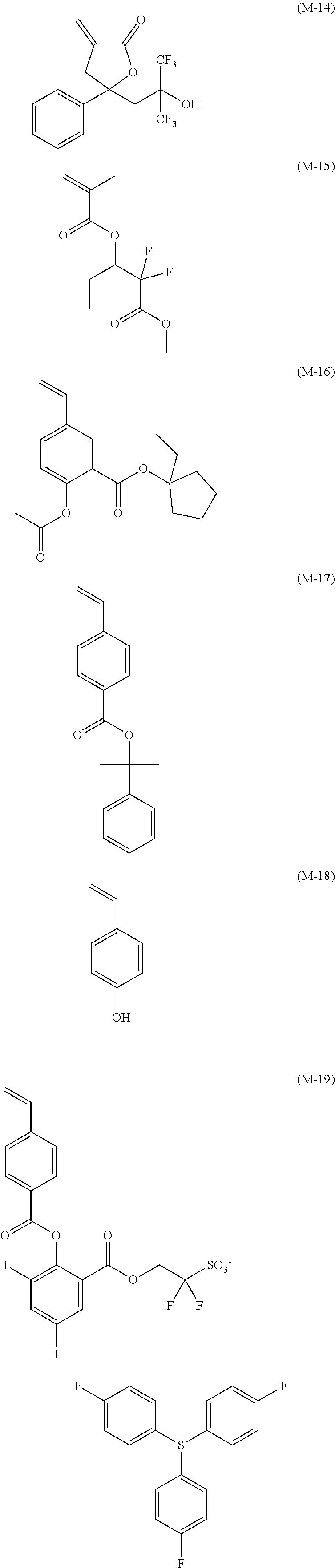

### Synthesis Example 2-1: Synthesis of Polymer (A-1)

The monomer (M-2) and the monomer (M-8) were dissolved in propylene glycol monomethyl ether (200 parts by mass) such that a molar ratio of the monomers became 55/45. Next, thereto was added as an initiator, 2,2′-azobis(methylisobutyrate) (10 mol %) to prepare a monomer solution. Propylene glycol monomethyl ether (100 parts by mass) was charged into an empty reaction vessel, and was heated to 85° C. with stirring. Next, the monomer solution prepared as described above was added dropwise over 3 hours, and then a thus resulting solution was further heated for 3 hours at 85° C. After completion of the polymerization reaction, the polymerization solution was cooled to room temperature. The polymerization solution thus cooled was added dropwise into n-hexane (1,000 parts by mass) to allow for solidification purification of the polymer. To the polymer thus recovered were added propylene glycol monomethyl ether (150 parts by mass), methanol (150 parts by mass), triethylamine (1.5 molar equivalent with respect to the using amount of the monomer (M-2)), and water (1.5 molar equivalent with respect to the using amount of the monomer (M-2)). A hydrolysis reaction was performed for 8 hours while reflux was allowed at a boiling point. After completion of the reaction, the solvent and the triethylamine were distilled away under reduced pressure, and a polymer thus obtained was dissolved in acetone (150 parts by mass). This solution was added dropwise into water (2,000 parts by mass) to permit solidification, and white powder thus generated was filtered off. Drying at 50° C. for 17 hours gave a white powdery polymer (A-1) with a favorable yield. The Mw of the polymer (A-1) was 5,900, and the Mw/Mn was 1.5.

### Synthesis Examples 2-2 to 2-18: Synthesis of Polymers (A-2) to (A-18)

Polymers (A-2) to (A-18) were synthesized in a similar manner to Synthesis Example 2-1 except that monomers of the type and the using amount shown in Table 1 below were used.

### Synthesis Example 2-19: Synthesis of Polymer (A-19)

In a similar manner to Synthesis Example 2-1 except that the step of hydrolysis with triethylamine was not performed in Synthesis Example 2-1, each monomer was combined and copolymerization thereof was allowed to proceed in the presence of a tetrahydrofuran (THF) solvent, followed by isolation and drying, to give a polymer (A-19) having a constitution as shown below. The constitution of each polymer thus obtained was confirmed by ¹H-NMR, and the Mw and the dispersity index (Mw/Mn) thereof were confirmed in accordance with the above-described GPC conditions.

The type and the proportion in use, and the Mw and the Mw/Mn of the monomer that gives each structural unit of each of the polymers (A) obtained in Synthesis Examples 2-1 to 2-19 are shown in Table 1 below. In Table 1 below, “-” indicates that the corresponding monomer was not used.

TABLE 1

| | | Monomer that | | Monomer that | | | | | |
|---|---|---|---|---|---|---|---|---|---|
| | | gives structural | | gives structural | | Monomer that gives | | | |
| | | unit (I) | | unit (II) | | other structural unit | | | |
| | | | using | | using | | using | | |
| | (A) | | amount | | amount | | amount | | |
| | Polymer | type | (mol %) | type | (mol %) | type | (mol %) | Mw | Mw/Mn |
| Synthesis | A-1 | M-8 | 45 | M-2 | 55 | — | — | 5,900 | 1.5 |
| Example 2-1 | | | | | | | | | |
| Synthesis | A-2 | M-7 | 45 | M-2 | 55 | — | — | 6,000 | 1.5 |
| Example 2-2 | | | | | | | | | |
| Synthesis | A-3 | M-9 | 45 | M-2 | 55 | — | — | 6,500 | 1.6 |
| Example 2-3 | | | | | | | | | |
| Synthesis | A-4 | M-9/M-10 | 35/10 | M-2 | 55 | — | — | 6,600 | 1.7 |
| Example 2-4 | | | | | | | | | |
| Synthesis | A-5 | M-11 | 45 | M-2 | 55 | — | — | 6,100 | 1.5 |
| Example 2-5 | | | | | | | | | |
| Synthesis | A-6 | M-12 | 45 | M-2 | 55 | — | — | 6,500 | 1.5 |
| Example 2-6 | | | | | | | | | |
| Synthesis | A-7 | M-9 | 40 | M-2 | 60 | — | — | 6,400 | 1.5 |
| Example 2-7 | | | | | | | | | |
| Synthesis | A-8 | M-9 | 50 | M-2 | 50 | — | — | 6,500 | 1.6 |
| Example 2-8 | | | | | | | | | |
| Synthesis | A-9 | M-9 | 60 | M-2 | 40 | — | — | 6,200 | 1.5 |
| Example 2-9 | | | | | | | | | |
| Synthesis | A-10 | M-8 | 45 | M-2/M-3 | 35/20 | — | — | 5,500 | 1.6 |
| Example 2-10 | | | | | | | | | |
| Synthesis | A-11 | M-8 | 45 | M-2/M-1 | 35/20 | — | — | 5,400 | 1.6 |
| Example 2-11 | | | | | | | | | |
| Synthesis | A-12 | M-8 | 45 | M-2/M-4 | 35/20 | — | — | 5,500 | 1.5 |
| Example 2-12 | | | | | | | | | |
| Synthesis | A-13 | M-8 | 45 | M-2 | 35 | M-5 | 20 | 5,800 | 1.5 |
| Example 2-13 | | | | | | | | | |
| Synthesis | A-14 | M-8 | 45 | M-2/M-6 | 35/20 | — | — | 6,000 | 1.5 |
| Example 2-14 | | | | | | | | | |
| Synthesis | A-15 | M-8 | 45 | M-2 | 35 | M-13 | 20 | 6,100 | 1.4 |
| Example 2-15 | | | | | | | | | |
| Synthesis | A-16 | M-8 | 45 | M-2 | 35 | M-14 | 20 | 6,200 | 1.6 |
| Example 2-16 | | | | | | | | | |
| Synthesis | A-17 | M-8/M-16 | 25/20 | M-2 | 55 | — | — | 6,400 | 1.6 |
| Example 2-17 | | | | | | | | | |
| Synthesis | A-18 | M-8/M-17 | 25/20 | M-2 | 55 | — | — | 6,600 | 1.6 |
| Example 2-18 | | | | | | | | | |
| Synthesis | A-19 | M-8 | 45 | M-18 | 45 | M-19 | 10 | 6,600 | 1.7 |
| Example 2-19 | | | | | | | | | |

### Synthesis of Polymer (F)

According to the following procedure, a polymer (F-1) to serve as the polymer (F) was synthesized. For synthesis of the polymer (F), the above monomer (M-8) and a compound (hereinafter, may be also referred to as “monomer (M-15)”) represented by the above formula (M-15) were used.

### Synthesis Example 3: Synthesis of Polymer (F-1)

The monomer (M-8) and the monomer (M-15) were dissolved in 2-butanone (100 parts by mass) such that a molar ratio of the monomers became 40/60. Thereto was added as an initiator, azobis(isobutyronitrile) (4 mol %) to prepare a monomer solution. 2-Butanone (50 parts by mass) was charged into an empty reaction vessel, and nitrogen was purged for 30 min. The interior of this reaction vessel was heated to 80° C., and the monomer solution was added dropwise thereto over 3 hours with stirring. After completion of the dropwise addition, the solution was further heated at 80° C. for 3 hours, and then the polymerization solution was cooled to 30° C. or lower. After the polymerization solution was transferred into a separatory funnel, hexane (150 parts by mass) was added to homogeneously dilute the polymerization solution. Furthermore, methanol (600 parts by mass) and water (30 parts by mass) were charged and mixed. After the mixture was left to stand for 30 min, the underlayer was recovered and the solvent was substituted with propylene glycol monomethyl ether acetate. In this manner, a 10% solution of the polymer (F-1) in propylene glycol monomethyl ether acetate was obtained. The polymer (F-1) had Mw=8,500, and Mw/Mn=1.8.

### Preparation of Radiation-Sensitive Composition

The acid generating agent (B), the acid diffusion control agent (C), and the organic solvent (D) used in preparation of the radiation-sensitive composition are shown below. In the following Examples and Comparative Examples, unless otherwise specified particularly, the term “parts by mass” means a value, provided that the mass of the polymer (A) used was 100 parts by mass, and the term “mol %” means a value, provided that the mol number of the acid generating agent (B) used accounted for 100 mol %.

### (B) Acid Generating Agent

Compounds (hereinafter, may be also referred to as “acid generating agents (B-1) to (B-10)”) represented by the following formulae (B-1) to (B-10) were used as the acid generating agents. The acid generating agents (B-1) to (B-8) and (B-10) correspond to the above compound (β).

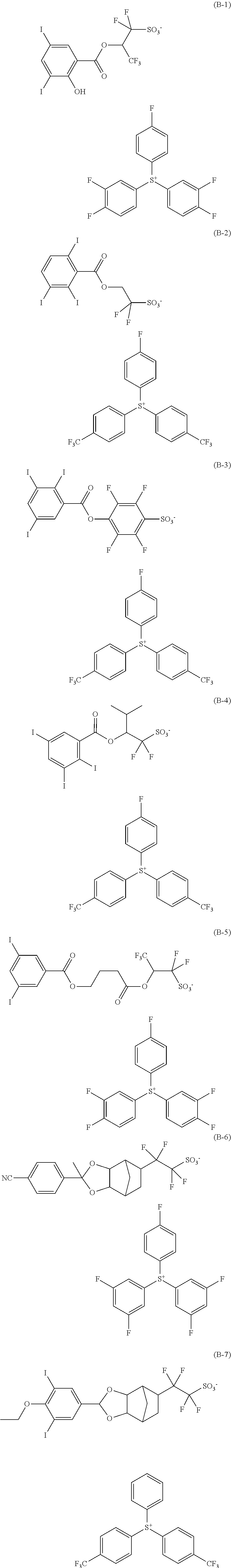

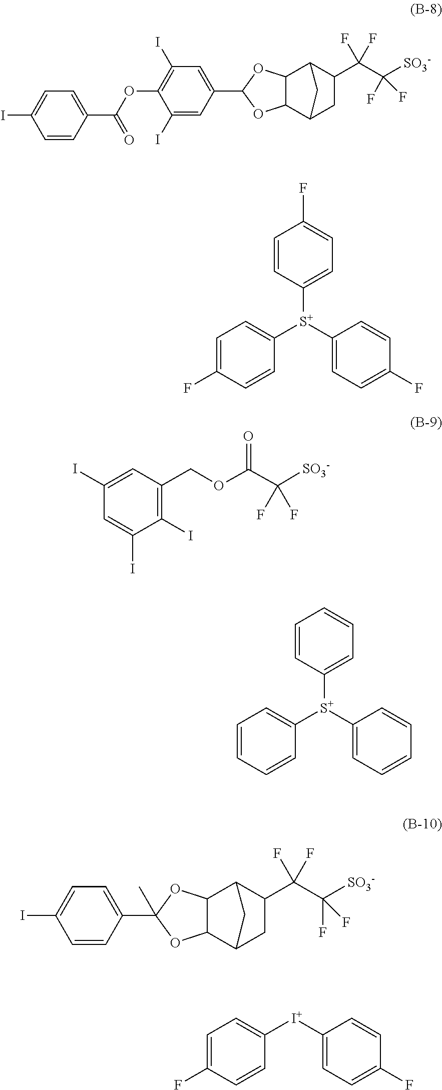

### (C) Acid Diffusion Control Agent

The compounds (Z-1) to (Z-15) and compounds represented by the following formulae (CC-1) to (CC-2) were used as the acid diffusion control agent (C).

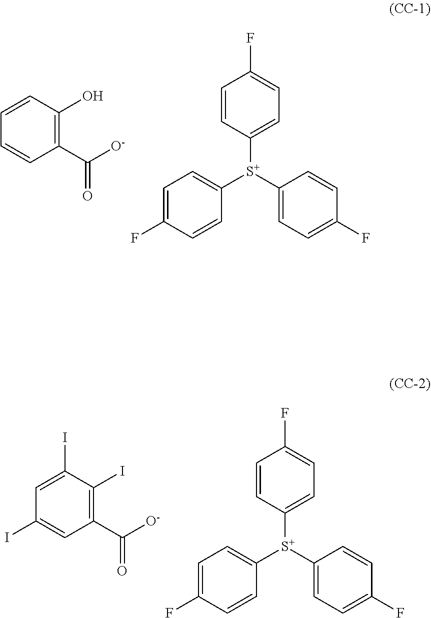 [SMILES: O=C([O-])c1ccccc1O ; Fc1ccc([S+](c2ccc(F)cc2)c2ccc(F)cc2)cc1 ; O=C([O-])c1cc(I)cc(I)c1I ; Fc1ccc([S+](c2ccc(F)cc2)c2ccc(F)cc2)cc1]

### (D) Organic Solvent

The following organic solvents were used as the organic solvent (D).
(D-1): propylene glycol monomethyl ether acetate
(D-2): propylene glycol monomethyl ether
(D-3): methyl 2-hydroxyisobutyrate

### Example 1: Preparation of Radiation-Sensitive Composition (R-1)

100 parts by mass of the polymer (A-1) to serve as the polymer (A), 3 parts by mass, in terms of a solid content, of the polymer (F-1) to serve as the polymer (F), 40 parts by mass of the acid generating agent (B-1) to serve as the acid generating agent (B), the compound (Z-1) to serve as the acid diffusion control agent (C) in an amount of 40 mol % with respect to the acid generating agent (B-1), and 1,500 parts by mass of (D-1) and 6,200 parts by mass of (D-2) to serve as the organic solvent (D) were mixed. A mix liquid thus obtained was filtered through a filter having a pore size of 0.20 m, whereby a radiation-sensitive composition (R-1) was prepared.

### Examples 2 to 44 and Comparative Examples 1 to 3: Preparation of Radiation-Sensitive Compositions (R-2) to (R-44) and (CR-1) to (CR-3)

Radiation-sensitive compositions (R-2) to (R-44) and (CR-1) to (CR-3) were prepared in a similar manner to Example 1 except that each component of the type and in the content shown in Table 2 below was used. It is to be noted that in Table 2 below, the “molar ratio Y/X” indicates a molar ratio of the content of the cation (Y) to the content of the anion (X), and “-” in this column indicates that at least one of the anion (X) and the cation (Y) was not contained, and thus the molar ratio was not calculated.

TABLE 2

| | | | | | | (B) Acid | | (C) Acid | | | | |
|---|---|---|---|---|---|---|---|---|---|---|---|---|
| | | (A) Polymer | | (F) Polymer | | generating agent | | diffusion | | | (D) Organic solvent | |
| | Radiation- | | content | | content | | content | control agent | | Molar | | content |
| | sensitive | | (parts by | | (parts by | | (parts by | | content | ratio | | (parts by |
| | composition | type | mass) | type | mass) | type | mass) | type | (mol %) | Y/X | type | mass) |
| Example 1 | R-1 | A-1 | 100 | F-1 | 3 | B-1 | 40 | Z-1 | 40 | 3.5 | D-1/D-2 | 1500/6200 |
| Example 2 | R-2 | A-1 | 100 | F-1 | 3 | B-1 | 40 | Z-2 | 40 | 3.5 | D-1/D-2 | 1500/6200 |
| Example 3 | R-3 | A-1 | 100 | F-1 | 3 | B-1 | 40 | Z-3 | 40 | 3.5 | D-1/D-2 | 1500/6200 |
| Example 4 | R-4 | A-1 | 100 | F-1 | 3 | B-1 | 40 | Z-4 | 40 | 3.5 | D-1/D-2 | 1500/6200 |
| Example 5 | R-5 | A-1 | 100 | F-1 | 3 | B-1 | 40 | Z-5 | 40 | 3.5 | D-1/D-2 | 1500/6200 |
| Example 6 | R-6 | A-1 | 100 | F-1 | 3 | B-1 | 40 | Z-6 | 40 | 3.5 | D-1/D-2 | 1500/6200 |
| Example 7 | R-7 | A-1 | 100 | F-1 | 3 | B-1 | 40 | Z-7 | 40 | 3.5 | D-1/D-2 | 1500/6200 |
| Example 8 | R-8 | A-1 | 100 | F-1 | 3 | B-1 | 40 | Z-8 | 40 | 3.5 | D-1/D-2 | 1500/6200 |
| Example 9 | R-9 | A-1 | 100 | F-1 | 3 | B-1 | 40 | Z-9 | 40 | 3.5 | D-1/D-2 | 1500/6200 |
| Example 10 | R-10 | A-1 | 100 | F-1 | 3 | B-1 | 40 | Z-10 | 40 | 3.5 | D-1/D-2 | 1500/6200 |
| Example 11 | R-11 | A-1 | 100 | F-1 | 3 | B-1 | 40 | Z-11 | 40 | 3.5 | D-1/D-2 | 1500/6200 |
| Example 12 | R-12 | A-1 | 100 | F-1 | 3 | B-1 | 40 | Z-12 | 40 | 2.5 | D-1/D-2 | 1500/6200 |
| Example 13 | R-13 | A-1 | 100 | F-1 | 3 | B-1 | 40 | Z-13 | 40 | 3.5 | D-1/D-2 | 1500/6200 |
| Example 14 | R-14 | A-1 | 100 | F-1 | 3 | B-2 | 40 | Z-1 | 40 | 3.5 | D-1/D-2 | 1500/6200 |
| Example 15 | R-15 | A-1 | 100 | F-1 | 3 | B-3 | 40 | Z-1 | 40 | 3.5 | D-1/D-2 | 1500/6200 |
| Example 16 | R-16 | A-1 | 100 | F-1 | 3 | B-4 | 40 | Z-1 | 40 | 3.5 | D-1/D-2 | 1500/6200 |
| Example 17 | R-17 | A-1 | 100 | F-1 | 3 | B-5 | 40 | Z-1 | 40 | 3.5 | D-1/D-2 | 1500/6200 |
| Example 18 | R-18 | A-1 | 100 | F-1 | 3 | B-6 | 40 | Z-1 | 40 | 3.5 | D-1/D-2 | 1500/6200 |
| Example 19 | R-19 | A-1 | 100 | F-1 | 3 | B-7 | 40 | Z-1 | 40 | 3.5 | D-1/D-2 | 1500/6200 |
| Example 20 | R-20 | A-1 | 100 | F-1 | 3 | B-8 | 40 | Z-1 | 40 | 3.5 | D-1/D-2 | 1500/6200 |
| Example 21 | R-21 | A-1 | 100 | F-1 | 3 | B-9 | 40 | Z-1 | 40 | 1.0 | D-1/D-2 | 1500/6200 |
| Example 22 | R-22 | A-1 | 100 | F-1 | 3 | B-1 | 50 | Z-1 | 40 | 3.5 | D-1/D-2 | 1500/6200 |
| Example 23 | R-23 | A-1 | 100 | F-1 | 3 | B-1 | 60 | Z-1 | 40 | 3.5 | D-1/D-2 | 1500/6200 |
| Example 24 | R-24 | A-2 | 100 | F-1 | 3 | B-1 | 40 | Z-1 | 40 | 3.5 | D-1/D-2 | 1500/6200 |
| Example 25 | R-25 | A-3 | 100 | F-1 | 3 | B-1 | 40 | Z-1 | 40 | 3.5 | D-1/D-2 | 1500/6200 |
| Example 26 | R-26 | A-4 | 100 | F-1 | 3 | B-1 | 40 | Z-1 | 40 | 3.5 | D-1/D-2 | 1500/6200 |
| Example 27 | R-27 | A-5 | 100 | F-1 | 3 | B-1 | 40 | Z-1 | 40 | 3.5 | D-1/D-2 | 1500/6200 |
| Example 28 | R-28 | A-6 | 100 | F-1 | 3 | B-1 | 40 | Z-1 | 40 | 3.5 | D-1/D-2 | 1500/6200 |
| Example 29 | R-29 | A-7 | 100 | F-1 | 3 | B-1 | 40 | Z-1 | 40 | 3.5 | D-1/D-2 | 1500/6200 |
| Example 30 | R-30 | A-8 | 100 | F-1 | 3 | B-1 | 40 | Z-1 | 40 | 3.5 | D-1/D-2 | 1500/6200 |
| Example 31 | R-31 | A-9 | 100 | F-1 | 3 | B-1 | 40 | Z-1 | 40 | 3.5 | D-1/D-2 | 1500/6200 |
| Example 32 | R-32 | A-10 | 100 | F-1 | 3 | B-1 | 40 | Z-1 | 40 | 3.5 | D-1/D-2 | 1500/6200 |
| Example 33 | R-33 | A-11 | 100 | F-1 | 3 | B-1 | 40 | Z-1 | 40 | 3.5 | D-1/D-2 | 1500/6200 |
| Example 34 | R-34 | A-12 | 100 | F-1 | 3 | B-1 | 40 | Z-1 | 40 | 3.5 | D-1/D-2 | 1500/6200 |
| Example 35 | R-35 | A-13 | 100 | F-1 | 3 | B-1 | 40 | Z-1 | 40 | 3.5 | D-1/D-2 | 1500/6200 |
| Example 36 | R-36 | A-14 | 100 | F-1 | 3 | B-1 | 40 | Z-1 | 40 | 3.5 | D-1/D-2 | 1500/6200 |
| Example 37 | R-37 | A-15 | 100 | F-1 | 3 | B-1 | 40 | Z-1 | 40 | 3.5 | D-1/D-2 | 1500/6200 |
| Example 38 | R-38 | A-16 | 100 | F-1 | 3 | B-1 | 40 | Z-1 | 40 | 3.5 | D-1/D-2 | 1500/6200 |
| Example 39 | R-39 | A-17 | 100 | F-1 | 3 | B-1 | 40 | Z-1 | 40 | 3.5 | D-1/D-2 | 1500/6200 |
| Example 40 | R-40 | A-18 | 100 | F-1 | 3 | B-1 | 40 | Z-1 | 40 | 3.5 | D-1/D-2 | 1500/6200 |
| Example 41 | R-41 | A-19 | 100 | F-1 | 3 | — | — | Z-1 | 40 | 3.5 | D-1/D- | 1500/3100/ |
| | | | | | | | | | | | 2/D-3 | 3100 |
| Example 42 | R-42 | A-1 | 100 | F-1 | 3 | B-1 | 40 | Z-14 | 40 | 3.5 | D-1/D- | 1500/3100/ |
| | | | | | | | | | | | 2/D-3 | 3100 |
| Example 43 | R-43 | A-1 | 100 | F-1 | 3 | B-1 | 40 | Z-15 | 40 | 3.5 | D-1/D- | 1500/3100/ |
| | | | | | | | | | | | 2/D-3 | 3100 |
| Example 44 | R-44 | A-1 | 100 | F-1 | 3 | B-10 | 40 | Z-1 | 40 | 3.5 | D-1/D- | 1500/3100/ |
| | | | | | | | | | | | 2/D-3 | 3100 |
| Comparative | CR-1 | A-1 | 100 | F-1 | 3 | B-1 | 40 | CC-1 | 40 | — | D-1/D-2 | 1500/6200 |
| Example 1 | | | | | | | | | | | | |
| Comparative | CR-1 | A-1 | 100 | F-1 | 3 | B-1 | 40 | CC-2 | 40 | — | D-1/D-2 | 1500/6200 |
| Example 2 | | | | | | | | | | | | |
| Comparative | CR-3 | A-1 | 100 | F-1 | 3 | B-9 | 40 | Z-12 | 40 | — | D-1/D-2 | 1500/6200 |
| Example 3 | | | | | | | | | | | | |

### Formation of Resist Pattern

By using a spin coater (“CLEAN TRACK ACT 12” available from Tokyo Electron Limited), the radiation-sensitive composition prepared as described above was applied on a 12-inch silicon wafer surface provided with an underlayer film (“AL412” available from Brewer Science, Inc.) having an average thickness of 20 nm formed thereon. A resist film having an average thickness of 40 nm was formed through PB carried out at 100° C. for 60 sec, followed by cooling at 23° C. for 30 sec. This resist film was irradiated with EUV light by using an EUV scanner (“NXE3300” available from ASML Co.: NA=0.33, irradiation condition: Conventional s=0.89). The resist film was subjected to PEB at 100° C. for 60 sec. Development was performed using a 2.38% by mass aqueous TMAH solution at 23° C. for 30 sec to form a positive-tone contact hole pattern with 50 nm pitch and 25 nm holes.

### Evaluations

The sensitivity, the CDU, and the storage stability were evaluated in accordance with the following methods. The results of the evaluations are shown in Table 3 below.

### Sensitivity

An exposure dose at which the contact hole pattern with 25 nm holes was formed in the above-described Formation of Resist Pattern was defined as an optimum exposure dose, and this optimum exposure dose was adopted as the sensitivity (mJ/cm²). The optimum exposure dose value being smaller indicates more favorable sensitivity. The sensitivity was evaluated to be: “A” (extremely favorable) in a case of less than 54 mJ/cm²; “B” (favorable) in a case of no less than 54 mJ/cm² and no greater than 56 mJ/cm²; “C” (somewhat favorable) in a case of no less than 56 mJ/cm² and no greater than 58 mJ/cm²; and “D” (poor) in a case of greater than 58 mJ/cm².

### CDU

The contact hole pattern with 25 nm holes was observed from above using a scanning electron microscope (“CG-4100” available from Hitachi High-Tech Corporation). Diameters in the contact hole pattern were measured at arbitrary 800 sites in total, and then a 3 Sigma value was determined from distribution of the measurement values and was defined as CDU (unit: nm). The CDU value being smaller indicates more favorable CDU, revealing less variance of the hole diameters over a long period of time. The CDU was evaluated to be: “A” (extremely favorable) in a case of the value being less than 3.9 nm; “B” (favorable) in a case of the value being no less than 3.9 nm and less than 4.1 nm; and “C” (poor) in a case of the value being no less than 4.1 nm.

### Storage Stability

The radiation-sensitive compositions prepared as described above were divided into samples to be stored at −15° C. and samples to be stored at 35° C., and each sample was stored for 2 weeks. Thereafter, the sensitivity was determined in accordance with the method described in the above section of Sensitivity. Based on a standard, i.e., the sensitivity of the sample stored at −15° C. for 2 weeks, the storage stability was evaluated on the sample stored at 35° C. for 2 weeks to be: “favorable” in a case in which a variation of the sensitivity was less than 1%; and “poor” in a case in which the variation of the sensitivity was no less than 10%.

TABLE 3

| | Radiation- | | | |
|---|---|---|---|---|
| | sensitive | | | Storage |
| | composition | Sensitivity | CDU | stability |
| Example 1 | R-1 | A | B | favorable |
| Example 2 | R-2 | A | B | favorable |
| Example 3 | R-3 | A | B | favorable |
| Example 4 | R-4 | A | B | favorable |
| Example 5 | R-5 | A | B | favorable |
| Example 6 | R-6 | C | B | favorable |
| Example 7 | R-7 | C | B | favorable |
| Example 8 | R-8 | C | B | favorable |
| Example 9 | R-9 | A | A | favorable |
| Example 10 | R-10 | A | A | favorable |
| Example 11 | R-11 | B | B | favorable |
| Example 12 | R-12 | B | B | favorable |
| Example 13 | R-13 | C | C | favorable |
| Example 14 | R-14 | A | B | favorable |
| Example 15 | R-15 | A | B | favorable |
| Example 16 | R-16 | A | B | favorable |
| Example 17 | R-17 | A | B | favorable |
| Example 18 | R-18 | B | B | favorable |
| Example 19 | R-19 | A | B | favorable |
| Example 20 | R-20 | B | B | favorable |
| Example 21 | R-21 | B | B | favorable |
| Example 22 | R-22 | A | A | favorable |
| Example 23 | R-23 | A | A | favorable |
| Example 24 | R-24 | A | B | favorable |
| Example 25 | R-25 | A | A | favorable |
| Example 26 | R-26 | A | A | favorable |
| Example 27 | R-27 | A | B | favorable |
| Example 28 | R-28 | A | B | favorable |
| Example 29 | R-29 | A | A | favorable |
| Example 30 | R-30 | A | A | favorable |
| Example 31 | R-31 | A | A | favorable |
| Example 32 | R-32 | A | B | favorable |
| Example 33 | R-33 | A | B | favorable |
| Example 34 | R-34 | A | B | favorable |
| Example 35 | R-35 | A | B | favorable |
| Example 36 | R-36 | A | B | favorable |
| Example 37 | R-37 | A | B | favorable |
| Example 38 | R-38 | A | B | favorable |
| Example 39 | R-39 | A | B | favorable |
| Example 40 | R-40 | A | B | favorable |
| Example 41 | R-41 | A | A | favorable |
| Example 42 | R-42 | A | A | favorable |
| Example 43 | R-43 | A | A | favorable |
| Example 44 | R-44 | A | A | favorable |
| Comparative | CR-1 | A | B | poor |
| Example 1 | | | | |
| Comparative | CR-2 | B | A | poor |
| Example 2 | | | | |
| Comparative | CR-3 | D | C | favorable |
| Example 3 | | | | |

Obviously, numerous modifications and variations of the present invention(s) are possible in light of the above teachings. It is therefore to be understood that within the scope of the appended claims, the invention(s) may be practiced otherwise than as specifically described herein.

What is claimed is:

1. A radiation-sensitive composition comprising:
a polymer, solubility of which in a developer solution is capable of being altered by an action of an acid;
an anion represented by formula (1); and
a radiation-sensitive onium cation comprising an aromatic ring and at least one fluorine atom or fluorine atom-containing group bonded to the aromatic ring, 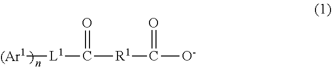
wherein, in the formula (1),
Ar¹ represents a group obtained by removing one hydrogen atom from a substituted or unsubstituted aromatic ring;
n is an integer of 1 to 3;
in a case in which n is no less than 2, a plurality of Ar¹s are identical or different;
R¹ represents a single bond or a substituted or unsubstituted divalent hydrocarbon group;
L¹ represents —O—, (*)ₙ—R²—O—, or —NR³—, wherein in a case in which n is no less than 2, L¹ represents (*)ₙ—R²O—;
* denotes a site bonding to Ar¹;
R² represents a substituted or unsubstituted hydrocarbon group having a valency of (n+1); and
R³ represents a hydrogen atom or a monovalent hydrocarbon group.

2. The radiation-sensitive composition according to claim 1, wherein R¹ in the formula (1) represents a single bond.

3. The radiation-sensitive composition according to claim 1, wherein L¹ in the formula (1) represents (*)ₙ—R²—O—.

4. The radiation-sensitive composition according to claim 1, wherein the aromatic ring that gives Ar¹ in the formula (1) is an aromatic ring in which at least one iodine atom is bonded to the aromatic ring.

5. The radiation-sensitive composition according to claim 1, wherein L¹ in the formula (1) represents (*)ₙ—R²—O—, and the aromatic ring that gives Ar¹ in the formula (1) is an aromatic ring in which at least one iodine atom is bonded to the aromatic ring.

6. A method of forming a resist pattern, the method comprising:
applying the radiation-sensitive composition according to claim 1 directly or indirectly on a substrate to form a resist film;
exposing the resist film; and
developing the resist film exposed.

7. A compound represented by formula (2), 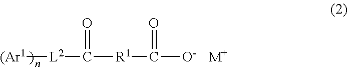
wherein, in the formula (2),
Ar¹ represents a group obtained by removing one hydrogen atom from a substituted or unsubstituted aromatic ring;
n is an integer of 1 to 3;
in a case in which n is no less than 2, a plurality of Ar¹s are identical or different;
R¹ represents a single bond or a substituted or unsubstituted divalent hydrocarbon group;
L² represents (*)ₙ—R²—O—;
R² represents a substituted or unsubstituted hydrocarbon group having a valency of (n+1);
* denotes a site bonding to Ar¹; and
M⁺ represents a monovalent cation.

8. The compound according to claim 7, wherein the aromatic ring that gives Ar¹ in the formula (2) is an aromatic ring in which at least one iodine atom is bonded to the aromatic ring.
